(19) United States
(12) Patent Application Publication
(10) Pub. No.: US 2026/0355279 A1
(43) Pub. Date: Nov. 12, 2026

(54) COMPOSITIONS AND METHODS FOR THE TREATMENT OF BONE LOSS

(71) Applicant: THE UNIVERSITY OF HONG KONG, Hong Kong (CN)

(72) Inventors: Ching Lung CHEUNG, Hong Kong (CN); Wai Chee Annie KUNG, Hong Kong (CN); Kathryn TAN, Hong Kong (CN); Kat Tik LAU, Hong Kong (CN); Suhas KRISHNAMOORTHY, Hong Kong (CN)

(21) Appl. No.: 19/488,076

(22) PCT Filed: May 10, 2024
(86) PCT No.: PCT/CN2024/092278

§ 371 Date: Nov. 26, 2025

Related U.S. Application Data
(60) Provisional application No. 63/504,531, filed on May 26, 2023.

(51) Int. Cl. A61K 38/18; A61K 31/7088; A61K 48/00; A61P 19/08

(52) U.S. Cl. CPC A61K 38/18; A61K 31/7088; A61K 48/0033; A61K 48/005; A61P 19/08

(57) ABSTRACT

GREM2 compositions and formulations thereof are described for use in treating or preventing one or more symptoms of a disease associated with loss of bone mineral density. Recombinant GREM2 have been shown to reduce bone resorption, reduce bone turnover markers, and increase bone strength. Methods of using the GREM2 compositions and formulations are also provided. Preferably, the pharmaceutical formulation containing GREM2 is administered via parenteral route to prevent or alleviate one or more symptoms of osteoporosis, osteopenia, bone fractures, and/or bone cancer.

### FIELD OF THE INVENTION

The disclosed invention generally relates to compositions and methods for treating conditions and diseases relating to bone loss.

### BACKGROUND OF THE INVENTION

Osteoporosis is a common bone disease characterized by low bone mineral density (BMD), deteriorated bone microarchitecture, and susceptibility to fractures (Sözen, T., et al., Eur J Rheumatol, 4(1): 46-56, (2017)). According to the World Health Organization, osteoporosis is defined by a BMD t-score equal to or less than −2.5. It has been estimated that more than 200 million people worldwide are suffering from osteoporosis, while more than 40% of the population is estimated to present with low bone mass, i.e., osteopenia (Sözen, T., et al., Eur J Rheumatol, 4(1): pgs. 46-56, (2017); Varacallo M, et al., StatPearls, (2022)). Given that osteoporosis is an age-related condition, it is expected that the prevalence of osteoporosis will increase continuously due to the expanding aging population worldwide.

Presently, different pharmacological agents are available to treat osteoporosis. For example, the first line of drugs used for treating osteoporosis or osteopenia include bisphosphonates, e.g., alendronate and zoledronate. However, long-term use of these drugs may lead to several adverse effects such as atypical fractures and osteonecrosis at the jaw (ONJ) (Salari, P. and M. Abdollahi, J Pharm Sci, 2012. 15(2): 305-17). Other than bisphosphonates, denosumab is also a common agent in treating osteoporosis. However, atypical fractures and ONJ are also known to be associated with long-term use of denosumab. The anabolic agents teriparatide, abaloparatide, and romosozumab are potentially associated with severe adverse drug reactions, such as osteosarcoma and adverse cardiac events. Thus, there is an urgent need of new medication to treat these diseases safely and effectively.

It is an object of the present invention to provide compositions to treat and/or prevent bone loss.

It is another object of the invention to provide a method of treating and/or preventing bone loss in a subject.

### BRIEF SUMMARY OF THE INVENTION

It has been discovered that Gremlin-2 (GREM2), a secreted BMP antagonist, is a negative regulator of osteoclastogenesis and bone resorption. Moreover, it has been discovered that GREM2 is associated with retention of bone mineral density and with decreased markers of bone loss in vivo.

Compositions and formulations containing soluble GREM2 protein (s) or nucleic acid (s) encoding a soluble protein are disclosed. The compositions and formulations are suitable for treating a disease, disorder, or condition associated with bone loss. In some forms, the compositions include one or more soluble proteins containing all or a functional portion of amino acids 22 to 168 of a Gremlin-2 (GREM2) protein, or a functional variant thereof. In some forms, the composition has an amino acid sequence of SEQ ID NO:1 or SEQ ID NO:2, or a functional fragment or variant thereof. For example, in some forms, the GREM2 protein (s) has a sequence similarity of about 70% to about 99% to SEQ ID NO:1 or SEQ ID NO:2. In some forms, the GREM2 protein (s) has a sequence similarity of about 70%, 75%, 80%, 85%, 90%, 92.5%, 95%, 99% to SEQ ID NO:1 or SEQ ID NO:2.

In some forms, the soluble protein contains a functional portion of amino acids 22 to 168 of a Gremlin-2 (GREM2) protein, of a variant thereof. In some forms, a GREM2 variant has a functional activity that is at least 20%, 30%, 40%, 50%, 60%, 70%, 80%, 90%, 95%, 100%, or more than 100%, such as 105%, 110% or more, of the GREM2 protein that is set forth by SEQ ID NO:1 or SEQ ID NO:2. Generally, a variant of any one of SEQ ID NO:1 and SEQ ID NO:2 is considered to have the function of directly or indirectly preventing or reducing bone loss in a subject in need thereof. For example, in some forms, the GREM2 variant has the function of increasing bone mineral density and reducing levels of bone turnover markers e.g., CTx and NTx markers.

In some forms, the functional portion or functional variant contains the native GREM2 protein antagonism to BMP2. In some forms, a variant of any one of SEQ ID NO:1 and SEQ ID NO:2 can have the function of directly or indirectly reducing the activity of BMP2 in the cell, if the variant has at least 20%, 30%, 40%, 50%, 60%, 70%, 80%, 90%, 95%, 100%, or more than 100%, such as 105%, 110% or more of the functional effect of the non-variant or native sequence from which it is derived. Variants of GREM2 generally should retain the consensus sequence CX₆QX₆CX₆NX₂CXGXCXSX₃PX₍₈₋₁₃₎CX₂CXPX₈TLXCX₍₁₅₋₁₈₎ CXC (SEQ ID NO: 4).

Functional GREM2 variants can retain the ability to bind to bone morphogenic protein 2 (BMP-2). Such binding is the basis for GREM2's BMP antagonism (Hsu et al., Molecular Cell 1:673-684 (1998)).

In some forms, the functional portion or functional variant of the GREM2 protein prevents or reduces bone loss by modulating the function of one or more other proteins. For example, in some forms, a variant of any one of SEQ ID NO:1 and SEQ ID NO:2 can have the function of directly or indirectly modulating the activity of one or more other proteins in the cell, if the variant has at least 20%, 30%, 40%, 50%, 60%, 70%, 80%, 90%, 95%, 100%, or more than 100%, such as 105%, 110% or more of the functional effect of the non-variant or native sequence from which it is derived. Exemplary proteins whose function can be modified by the GREM2 proteins and variants thereof include but are not limited to GDF8 (Growth Differentiation Factor-8), TGF-β (Transforming growth factor beta), and SMAD (Suppressor of Mothers against Decapentaplegic) such as SMAD1, SMAD2, SMAD3, SMAD4, SMAD5, SMAD6, SMAD7, and/or SMAD8.

In some forms, the compositions include one or more isolated nucleic acid (s) encoding a soluble protein containing a functional GREM2 protein or functional variant thereof, as disclosed herein, preferably, in a vector for delivery and expression in cells, preferably mammalian cells. Increased expression of GREM2 is effective to increase bone mineral density and reduce levels of bone turnover markers e.g., CTx and NTx markers.

Pharmaceutical formulations including the GREM2 protein (s) or nucleic acid (s), and a pharmaceutically acceptable carrier are also provided. In some forms, the pharmaceutical is lyophilized, or in the form of liquid or powder. Kits including the peptide are also described. Typically, the kits include one or more single unit dose of the peptide and instructions on how the dose is to be administered for treatment of one or more symptoms associated with bone loss in a subject.

Methods of using the disclosed compositions and formulations including, but not limited to, GREM2 proteins, nucleic acids and small molecules are provided. Methods of treatment including cells and other therapeutic agents including GREM2 peptides, nucleic acids, and/or small molecules are described. Generally, the method involves treating a subject (e.g., a human) having a disease, disorder, or condition by administering to the subject an effective amount of a pharmaceutical composition including genetically modified GREM2 peptides, nucleic acids, and/or small molecules. In some embodiments, the methods administer a pharmaceutical composition to express recombinant GREM2 proteins to a subject (e.g., a human) having a disease, disorder, or condition in an amount effective to treat the disease, disorder, or condition. For example, in some embodiments, the methods treat a disease or disorder associated with an elevated expression of BMP by administering to the subject an effective amount of a pharmaceutical composition including recombinant GREM2 peptides and/or proteins.

In some forms, the disease and/or disorder to be treated includes but is not limited to pre-osteoporosis, osteoporosis, osteopenia, bone fractures, and bone cancer. The disclosed compositions and methods can also be used to treat bone loss associated with various diseases. Such diseases include, for example, Rheumatoid Arthritis, Diabetes Mellitus type 1, Hyperthyroidism, Hyperparathyroidism, Celiac disease, Inflammatory bowel diseases such as Crohn's disease and ulcerative colitis. Particular conditions which can be treated using the disclosed compositions and methods include dysplasias, wherein growth or development of bone is abnormal and a wide variety of causes of osteopenia, osteoporosis and bone loss. Representative examples of such conditions include achondroplasia, cleidocranial dysostosis, enchondromatosis, fibrous dysplasia, Gaucher's Disease, hypophosphatemia rickets, Marfan's syndrome, multiple hereditary exostoses, neurofibromatosis, osteogenesis imperfecta, osteopetrosis, osteopoikilosis, sclerotic lesions, pseudoarthrosis, and pyogenic osteomyelitis, periodontal disease, anti-epileptic drug induced bone loss, primary and secondary hyperparathyroidism, familial hyperparathyroidism syndromes, weightlessness induced bone loss, osteoporosis in men, postmenopausal bone loss, osteoarthritis, renal osteodystrophy, infiltrative disorders of bone, oral bone loss, osteonecrosis of the jaw, juvenile Paget's disease, melorheostosis, metabolic bone diseases, mastocytosis, sickle cell anemia/disease, organ transplant related bone loss, kidney transplant related bone loss, systemic lupus erythematosus, ankylosing spondylitis, epilepsy, juvenile arthritis, thalassemia, mucopolysaccharidoses, Fabry disease, turner syndrome, Down Syndrome, Klinefelter Syndrome, leprosy, Perthes' Disease, adolescent idiopathic scoliosis, infantile onset multi-system inflammatory disease, Winchester Syndrome, Menkes Disease, Wilson's Disease, ischemic bone disease (such as Legg-Calve-Perthes disease, regional migratory osteoporosis), anemic states, conditions caused by steroids, glucocorticoid-induced bone loss, heparin-induced bone loss, bone marrow disorders, scurvy, malnutrition, calcium deficiency, idiopathic osteopenia or osteoporosis, congenital osteopenia or osteoporosis, alcoholism, chronic liver disease, postmenopausal state, chronic inflammatory conditions, rheumatoid arthritis, inflammatory bowel disease, ulcerative colitis, inflammatory colitis, Crohn's disease, oligomenorrhea, amenorrhea, pregnancy, diabetes mellitus, hyperthyroidism, thyroid disorders, parathyroid disorders, Cushing's disease, acromegaly, hypogonadism, immobilization or disuse, reflex sympathetic dystrophy syndrome, regional osteoporosis, osteomalacia, bone loss associated with joint replacement, HIV associated bone loss, bone loss associated with loss of growth hormone, bone loss associated with cystic fibrosis, fibrous dysplasia, chemotherapy associated bone loss, tumor induced bone loss, cancer-related bone loss, hormone ablative bone loss, multiple myeloma, drug-induced bone loss, anorexia nervosa, disease associated facial bone loss, disease associated cranial bone loss, disease associated bone loss of the jaw, disease associated bone loss of the skull, and bone loss associated with space travel. Further conditions relate to bone loss associated with aging, including facial bone loss associated with aging, cranial bone loss associated with aging, jawbone loss associated with aging, and skull bone loss associated with aging.

In some forms, the GREM2 compositions and formulations are administered to a subject in need to reduce one or more symptoms associated with bone loss, e.g., decreased bone mineral density, decreased bone strength, and decreased bone volume/tibial volume ratio (BV/TV). In some forms, the disease, disorder, or condition can be associated with an elevated expression or specific expression of a protein e.g., bone morphogenetic protein. For example, the GREM2 compositions can be administered to a subject in need to reduce the expression of one or more bone turnover markers.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIGS. 1A and 1B show TRAP staining of osteoclasts using different concentrations of GREM2. FIG. 1A is a micrograph of representative TRAP-staining results for GREM2 treatment at different concentrations. FIG. 1B is a dot plot showing quantification of TRAP staining of RAW264.7 with or without GREM2 treatment. **p<0.01, ***p<0.001 Student's t-test compared to the control. All values are given as the mean±SEM.

FIGS. 2A and 2B show results from a calcium phosphate surface resorption assay. FIG. 2A is a micrograph showing osteoclastic resorption assay of RAW264.7 without rank1, with rank1, and with both rank1 and GREM2 treatments. FIG. 2B is a bar graph of the total resorption area (%).

FIGS. 3A and 3B are dot plots showing the BMD- (FIG. 3A) and Bone volume to Tibia volume ratio (BV/TV) (FIG. 3B) in the GREM2 group and control group of healthy mice 4 weeks after treatment. The difference between the GREM2 group and the control group is ~14% in FIG. 3A and ~28% in FIG. 3B.

FIGS. 4A and 4B are dot plots showing BMD (FIG. 4A) and BV/TV (FIG. 4B) in the GREM2 group and control group of ovariectomized (OVX) mice 4 weeks after treatment.

### DETAILED DESCRIPTION OF THE INVENTION

The compositions and methods disclosed herein are based on the identification of Gremlin-2 (GREM2), an antagonist of bone morphogenetic protein (BMP), as a negative regulator of osteoclastogenesis and bone resorption. Gremlin 2 (GREM2) is a member of the DAN family Bone morphogenetic protein (BMP) antagonist and is a secreted BMP antagonist (Cheung, et al., The Journal of Clinical Endocrinology & Metabolism, 2013. 98(9):E1557-E1561). In a gene expression profiling study, GREM2 was one of the 6 genes that significantly up regulated by at least 2 folds during osteoblast differentiation in MC3T3 cells (Zamurovic, N., et al., J Biol Chem, 2004. 279(36):37704-15). The other 5 genes are: TGF-β1 (Transforming growth factor-β1), TGF-β3 (Transforming growth factor-β), PDGF (αPlatelet-derived growth factor, α), IGFBP10 (Insulin-like growth factor-binding protein 10), Activin (Inhibin β-A). These other genes are other members of the TGF-beta superfamily, are involved in BMP signaling pathways, but are not BMP antagonists.

GREM2 expression increases during early osteoblast differentiation and remains unchanged throughout the rest of bone differentiation and mineralization (Ideno, H., et al., Experimental Cell Research, 315(3): 474-484 (2009)). Although GREM2 expression increases during osteoblast differentiation, overexpression of GREM2 suppresses bone differentiation in murine pre-osteoblast cells and human bone marrow-derived mesenchymal stem cells (Wang, et al., J Cell Biochem, 118(2): 286-297 (2017)).

Mechanistic studies in the non-limiting Examples show that increased GREM2 expression is positively associated with increased bone mineral density (BMD), and that GREM2 is a negative regulator of osteoclastogenesis and bone resorption. Moreover, the Examples demonstrate that serum level of GREM2 is associated with increased BMD and reduced levels of bone turnover markers in humans. Finally, the Examples demonstrate that GREM2 treatment increases BMD and the ratio of Bone volume to Tibia volume (BV/TV) in both healthy and ovariectomized mice. Thus, compositions and formulations containing GREM2 are disclosed. The compositions are effective for treating and/or preventing conditions and diseases associated with bone loss, e.g., osteoporosis and osteopenia.

### I. DEFINITIONS

As used herein, the term “gene” refers to a nucleic acid (e.g., DNA or RNA) sequence that includes coding sequences necessary for the production of a polypeptide, RNA (e.g., including, but not limited to, mRNA, tRNA and rRNA) or precursor. The polypeptide, RNA, or precursor can be encoded by a full-length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation.

As used herein, “mammal” includes both humans and non-humans and includes but is not limited to humans, non-human primates, canines, felines, murines, bovines, equines, and porcines.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

As used herein, an “expression control sequence” is a DNA sequence that controls and regulates the transcription and/or translation of another DNA sequence.

As used herein, the term “treating” includes alleviating the symptoms associated with a specific disorder or condition and/or preventing or eliminating the symptoms.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, “transformed” and “transfected” encompass the introduction of a nucleic acid (e.g., a vector) into a cell by a number of techniques known in the art.

“Effective amount” and “therapeutically effective amount,” used interchangeably, as applied to the nanoparticles, therapeutic agents, and pharmaceutical compositions described herein, mean the quantity necessary to render the desired therapeutic result. For example, an effective amount is a level effective to treat, cure, or alleviate the symptoms of a disease for which the composition and/or therapeutic agent, or pharmaceutical composition, is/are being administered.

The term “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “pharmaceutically acceptable salt” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; etc.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, compositions including therapeutic agents may inhibit or reduce one or more markers of a disease or disorder in a subject by about 10%, 20%, 30%, 40%, 50%, 75%, 85%, 90%, 95%, or 99% from the activity and/or quantity of the same marker in subjects that did not receive, or were not treated with the compositions. In some forms, the inhibition and reduction are compared according to the level of mRNAs, proteins, cells, tissues and organs.

The terms “treating” or “retarding development of” in the context of a disease or disorder mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with a coronavirus infection are mitigated or eliminated, including, but are not limited to, reducing and/or inhibiting the syncytial formation and lung damage, increasing the quality of life of those suffering from the disease, decreasing the dose of other medications required to treat the disease, delaying the progression of the disease, and/or prolonging survival of individuals.

The term “biodegradable” generally refers to a material that will degrade or erode under physiologic conditions to smaller units or chemical species that are capable of being metabolized, eliminated, or excreted by the subject. The degradation time is a function of composition and morphology.

The term “fragment” means a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids.

The terms “isolated,” “purified,” or “biologically pure” mean material that is free to varying degrees from components which normally accompany it as found in its native state. “Isolate” denotes a degree of separation from original source or surroundings. “Purify” denotes a degree of separation that is higher than isolation.

“Variant” refers to a polypeptide or polynucleotide that differs from a reference polypeptide or polynucleotide but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. The term “polypeptides” includes proteins and fragments thereof. By “protein” or “polypeptide” or “peptide” is meant any chain of more than two natural or unnatural amino acids, regardless of post-translational modification (e.g., glycosylation or phosphorylation), constituting all or part of a naturally occurring or non-naturally occurring polypeptide or peptide, as is described herein. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, a protein is “soluble” when it lacks any transmembrane domain or protein domain that anchors or integrates the polypeptide into the membrane of a cell expressing such polypeptide. Soluble proteins lack additional hydrophobic sequences and are translocated through a translocon (e.g., a protein channel), to complete their folding and modification in the lumen of the endoplasmic reticulum.

The term “dosage regime” refers to drug administration regarding formulation, route of administration, drug dose, dosing interval and treatment duration.

Recitation of ranges of values herein are merely intended to serve as a shorthand method of referring individually to each separate value falling within the range, unless otherwise indicated herein, and each separate value is incorporated into the specification as if it were individually recited herein.

### II. COMPOSITIONS

Compositions that increase the expression of GREM2 are provided for use in the disclosed methods. The compositions generally include one or more GREM2 proteins, or nucleic acids or small molecules that that encode or increase the expression of GREM2. In some forms, the compositions include a soluble protein containing all or a functional portion of amino acids 22 to 168 of a Gremlin-2 (GREM2) protein or a functional variant thereof. In some forms, the compositions include a nucleic acid encoding the soluble protein all or a functional portion of amino acids 22 to 168 of a Gremlin-2 (GREM2) protein. The compositions are effective at interfering with or inhibiting the physiological action of Bone Morphogenetic Protein-2 (BMP2).

### A. GREM2 Proteins

Compositions of GREM2 proteins are provided for administration to a subject. The human Gremlin-2 (GREM2) amino acid sequence (UniProtKB-Q9H772_HUMAN) is as follows:

(SEQ ID NO: 1)
MFWKLSLSLFLVAVLVKVAEARKNRPAGAIPSPYKDGSSNNSERWQHQIK
EVLASSQEALVVTERKYLKSDWCKTQPLRQTVSEEGCRSRTILNRFCYGQ
CNSFYIPRHVKKEEESFQSCAFCKPQRVTSVLVELECPGLDPPFRLKKIQ
KVKQCRCMSVNLSDSDKQ.

Generally, the composition contains a soluble protein, wherein the soluble protein includes one or more GREM2 polypeptides. In some forms, the one or more GREM2 polypeptides include any naturally occurring polypeptide of a DAN (differential screening selected gene aberrative in neuroblastoma) family member, as well as any variants thereof (variants, fragments, etc.) that retain useful activity (e.g., fusion proteins, and peptidomimetic forms). For example, the GREM2 polypeptide has a sequence that is at least about 70% identical to the sequence of the GREM2 polypeptide, for example at least 70%, 75%, 80%, 85%, 90%, 95%, 97%, 99% sequence similarity. Polypeptides derived from any known sequence of GREM2 having lesser than 70% and more than 99% identity are included. Preferably, the GREM2 proteins prevent or reduce bone loss. Also preferably, the GREM2 proteins promote bone growth and bone mineralization. In some forms, the GREM2 polypeptides can either directly or indirectly antagonize the function of BMP2, e.g., by binding to BMP2 or by binding to a BMP receptor. Examples of GREM2 polypeptides include human Gremlin-2 (GREM2) (SEQ ID NO:1) and soluble proteins containing functional fragments of a Gremlin-2 protein e.g., amino acids 22 to 168 of a Gremlin-2 (GREM2) protein (SEQ ID NO:2).

In some forms, the soluble protein includes a GREM2 protein variant. In some forms, the composition includes GREM2 variants having between about 70% to about 990 sequence similarity to the amino acid sequence of SEQ ID NO:1 or SEQ ID NO:2. In some forms, the composition includes GREM2 variants at least about 70%, 75%, 80%, 85%, 90%, to 95% identical to the amino acid sequence of SEQ ID NO:1 or SEQ ID NO:2. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides disclosed herein and still obtain a molecule having similar characteristics as the polypeptide (e.g., a conservative amino acid substitution). For example, certain amino acids can be substituted for other amino acids in a sequence, without appreciable loss of activity. Since it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, and antigens. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly when the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

Amino acid substitutions are generally based on the relative similarity of the amino acid side-chain substituents, for example, their hydrophobicity, hydrophilicity, charge, and size. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). The polypeptides can include variants having about 50%, 60%, 70%, 80%, 90%, and 95% sequence identity to the polypeptide of interest.

“Identity” and “similarity” can be readily calculated by known methods, such as those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo and Lipman, SIAM J Applied Math, 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations include at least one amino acid deletion, substitution, including conservative and non-conservative substitution, or insertion, wherein the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from the total number of amino acids in the reference polypeptide.

In some forms, the GREM2 protein is a recombinant protein derived from GREM2 or a fragment of GREM2. SEQ ID NO:2 is an exemplary recombinant GREM2 protein:

(SEQ ID NO: 2)
RKNRPAGAIPSPYKDGSSNNSERWQHQIKEVLASSQEALVVTERKYLKSD
WCKTQPLRQTVSEEGCRSRTILNRFCYGQCNSFYIPRHVKKEEESFQSCA
FCKPQRVTSVLVELECPGLDPPFRLKKIQKVKQCRCMSVNLSDSDKQ

In some forms, a GREM2 variant has a functional activity that is at least 20%, 30%, 40%, 50%, 60%, 70%, 80%, 90%, 95%, 100%, or more than 100%, such as 105%, 110% or more, of the GREM2 protein that is set forth by SEQ ID NO:1 or SEQ ID NO:2. Generally, a variant of any one of SEQ ID NO:1 and SEQ ID NO:2 is considered to have the function of directly or indirectly preventing or reducing bone loss in a subject in need thereof. For example, in some forms, the GREM2 variant has the function of increasing bone mineral density and reducing levels of bone turnover markers e.g., CTx and NTx markers.

In some forms, the soluble protein contains a functional portion of amino acids 22 to 168 of a Gremlin-2 (GREM2) protein, of a variant thereof. In some forms, the functional portion or functional variant contains the native GREM2 protein antagonism to BMP2. In some forms, a variant of any one of SEQ ID NO:1 and SEQ ID NO:2 is considered to have the function of directly or indirectly reducing the activity of BMP2 in the cell, if the variant has at least 20%, 30%, 40, 50, 60, 70%, 80%, 90%, 95%, 100%, or more than 100%, such as 105%, 110% or more of the functional effect of the non-variant or native sequence from which it is derived. Variants of GREM2 generally should retain the consensus sequence

(SEQ ID NO: 4)
CX₆QX₆CX₆NX₂CXGXCXSX₃PX₍₈₋₁₃₎CX₂CXPX₈TLXCX₍₁₅₋₁₈₎CXC.

Functional GREM2 variants can bind to bone morphogenic protein 2 (BMP-2). Such binding is the basis for GREM2's BMP antagonism (Hsu et al., Molecular Cell 1:673-684 (1998)).

In some forms, the functional portion or functional variant of the GREM2 protein prevents or reduces bone loss by modulating the function of one or more other proteins. For example, in some forms, a variant of any one of SEQ ID NO:1 and SEQ ID NO:2 can have the function of directly or indirectly modulating the activity of one or more other proteins in the cell, if the variant has at least 20%, 30%, 40%, 50%, 60%, 70%, 80%, 90%, 95%, 100%, or more than 100%, such as 105%, 110% or more of the functional effect of the non-variant or native sequence from which it is derived. Exemplary proteins whose function can be modified by the GREM2 proteins and variants thereof include but are not limited to GDF8 (Growth Differentiation Factor-8), TGF-β (Transforming growth factor beta), and SMAD (Suppressor of Mothers against Decapentaplegic) such as SMAD1, SMAD2, SMAD3, SMAD4, SMAD5, SMAD6, SMAD7, and/or SMAD8.

As used herein, “native protein” broadly refers to a molecule having an amino acid sequence that may be isolated from an organism without modification by recombinant DNA techniques or other methods. Thus, “native protein” includes naturally occurring alleles and variants of the protein.

Typically, the GREM2 protein variant includes a molecule or sequence that is modified from a native protein but still retains the pharmacological activity of interest. Thus, the term “protein variant” contains a molecule or sequence in which non-native residues substitute for native residues, non-native residues are added, or native residues are deleted. Any native residue may be removed because it provides structural features or biological activity that are not required for the pharmacological activity of interest of the fusion molecules of the present invention. Thus, the term “protein variant” includes a molecule or sequence that lacks one or more native protein sites or residues that affect or are involved in any number of cellular processes including but not limited to (1) intracellular signaling, (2) incompatibility with a selected host cell (3) N-terminal heterogeneity upon expression in a selected host cell, (4) glycosylation, (5) interaction with other proteins (e.g., dimerization domains), (6) binding to a receptor or other protein that does not affect the pharmacological activity of interest, or (7) antibody-dependent cellular cytotoxicity (ADCC).

A functionally active fragment of a GREM2 protein can be obtained by screening a recombinantly produced polypeptide from the corresponding fragment of the nucleic acid encoding the GREM2 protein. In addition, fragments can be chemically synthesized using techniques known in the art, such as conventional Merrifield solid phase f-Moc or t-Boc chemistry. Fragments can be produced (recombinant or by chemical synthesis) and tested to identify such peptidyl fragments that can function as antagonists (inhibitors) of BMP-mediated signaling.

Functional variants can be generated by modifying the structure of the GREM2 protein for purposes such as enhanced therapeutic efficacy or stability (e.g, ex vivo shelf life and resistance to in vivo proteolysis). If selected to retain BMP antagonism, such modified GREM2 proteins are considered functional equivalents of naturally occurring GREM2 proteins. Modified GREM2 proteins can also be produced, for example, by amino acid substitution, deletion, or addition. For example, single replacement of leucine with isoleucine or valine, single replacement of aspartic acid with glutamic acid, single replacement of threonine with serine, or similar replacement of amino acids with structurally related amino acids (e.g., conservation) It is reasonable to expect that the (mutational) will not have a significant effect on the biological activity of the resulting molecule. Conservative replacements are those that take place within a family of amino acids that are associated with their side chains. Whether a change in the amino acid sequence of an GREM2 protein results in a functional homolog is readily determined by assessing the ability of the variant GREM2 protein to produce a response in a manner similar to the wild-type GREM2 protein.

Exemplary variants include one or more peptides having an amino acid sequence that is at least about 70%, 75%, 80%, 85%, 90%, 95%, or 99% identical to one or more of the respective amino acid sequences of SEQ ID NO:1 or SEQ ID NO:2. For example, variants can include one or more peptides having an amino acid sequence that is at least about 70%, 75%, 80%, 85%, 90%, 95%, or 99% identical to one or more of the respective amino acid sequences of SEQ ID NO:1 and SEQ ID NO:2. In some forms, the variants include one or more peptides having an amino acid sequence that is at least about 70%, 75%, 80%, 85%, 90%, 95%, or 99% identical to the amino acid sequence of SEQ ID NO:1. In other forms, the variants include one or more peptides having an amino acid sequence that is at least 80% identical to the amino acid sequence of SEQ ID NO:1. In other forms, the variants include one or more peptides having an amino acid sequence that is at least 80% identical to the amino acid sequence of SEQ ID NO:2.

The disclosed soluble proteins containing a GREM2 protein or a functional variant thereof, can have a length of up to 150, 200, 250, 300, 400, 500, 1000 or 2000 residues. In particular forms, the soluble proteins can have a length of at least 50, 60, 70, 80, 90, 100, 120, or 140 residues. In further embodiments, the soluble proteins can have a length of 50 to 200 residues, 50 to 100 residues, 50 to 90 residues, 50 to 80 residues, 50 to 70 residues, or 50 to 60 residues. As used herein, the term “residue” refers to an amino acid or amino acid analog.

### B. Nucleic Acids

Disclosed compositions can include isolated nucleic acids encoding any of the GREM2 proteins e.g., soluble GREM2 proteins including fragments, and functional variants disclosed herein. In some forms, the nucleic acid is a recombinant nucleic acid. In some forms. the nucleic acid may be single-stranded or double-stranded. Such a nucleic acid may be a DNA molecule or an RNA molecule. These nucleic acids can be used, for example, in methods for making GREM2 proteins or as direct therapeutics (e.g., in gene therapy approaches).

In some forms, the nucleic acids encode a GREM2 protein, or a functional fragment thereof. Increased expression of the GREM2 is effective to antagonize BMP2 activity, thereby increasing bone mineral density and reducing levels of bone turnover markers e.g., CTx and NTx markers.

The GREM2 nucleic acid sequence contains 4176 bp as detailed below (NCBI Reference: NM_022469.4):

(SEQ ID NO: 3)
AGAGCTAAGTCTCCGGGCTCCTTTCACATCTGCAGGAGCAGCCAGCCTGGGACCGCGCGGCGCGGGGCGC
GCTCTCGCCTCTCCTGCACCCTCAGCCGGCGCGCTTCTCTTATGGGCGTCTGCTGCAGTCTGGCTGCGGT
CGAACTGAAAGCGGCGGCGGGAGACCAAACTTAGACCCCGCTGTGGACTAGAGAACTCAGAGAAGGCAGA
GGGAGAGGGAGAGAGAGAGAGAGAAGGGACCCGAGGAGGAGGCTTCCATCACGTCATTGCAGGATGTTCT
GGAAGCTTTCCCTGTCCTTGTTCCTGGTGGCGGTGCTGGTGAAGGTGGCGGAAGCCCGGAAGAACCGGCC
GGCGGGCGCCATCCCCTCGCCTTACAAGGACGGCAGCAGCAACAACTCGGAGAGATGGCAGCACCAGATC
AAGGAGGTGCTGGCCTCCAGCCAGGAGGCCCTGGTGGTCACCGAGCGCAAGTACCTCAAGAGTGACTGGT
GCAAGACGCAGCCGCTGCGGCAGACGGTGAGCGAGGAGGGCTGCCGGAGCCGCACCATCCTCAACCGCTT
CTGCTACGGCCAGTGCAACTCCTTCTACATCCCGCGGCACGTGAAGAAGGAGGAGGAGTCCTTCCAGTCC
TGCGCCTTCTGCAAGCCCCAGCGCGTCACCTCCGTCCTCGTGGAGCTCGAGTGCCCCGGCCTGGACCCAC
CCTTCCGACTCAAGAAAATCCAGAAGGTGAAGCAGTGCCGGTGCATGTCCGTGAACCTGAGCGACTCGGA
CAAGCAGTGAGCGCCGGGCCGGACGCAGCTCAGCCCCGCGCGCGCCCGGCCGCTGGGTGGCGCCGCCGCC
GCCTCTGTCCCTGCCCTCCGAGCCCACTGTCACGCTGCCTGGTGTTCCCCCTCAGCAGCAAGCACTTCTC
TTAGGGCTGACGGTGTCCTTGTCACAGACGTGGATCGCAAGTGGAGCTTTTGCTGATGTGTTCCTGACCG
ACCCCGGGTCCCACCTGTGCCCCCTAAACCAGGCGGTGGATCAGGCCCGAAGGAAAATGCTGGAAAACCA
CCACCGCCACCCAGCAGTCAGAGAGGACCTTTCGAGGTGATGCACTGTAAATCCGGCATTGATGTTTCCA
CCATGTGGAAAAGCAAGGATTTCCCTGGCCCCGCCCTGCACTCCAGCTCGCCCCTTTCCCGCGCGAAGTG
AAGGATGCGTCACTTTTCTGAGCGGCCCCTCAGACCTCTCCTGCTGGTCCCTGGACCTCTGGCTGGGAGC
GCTCATTCTTGTGACTGGCAGGCTGCCCTCGGTCGCTGCTGTGATGAGGATATGTGCAACCTCCATAAAT
ATGTCGGTGGACGGGGCGTCTCCTAATAAACCTGATACCAAGAAGACAACTTGAGCCACTTGGATCCCGA
AGTGGGCTTCCCGGGAAACCTCGAGCAGAGCAGGCTACACAATCCCCCATCCTCAAGTCCCCACCGCCTC
TTCTTCCTCACTGTCCCCGCCCCCTATTTGTGGACTAAAGATGAACTCTGGTGTGCATGCTATTATTGCT
GCAATTAGAATATTATCACACACAAAGGTCTCTATATTAACGCTGGTTTTATAAGTGACCGTATATTGTA
AAGAGCTGTTAAAAAGTATTATAGACCTATTCATGTATTATTTCACATGTTTTGACTCTGGCTTGCAGCA
CCATTCGGAGTAAGGATGACAGCAGGCCCAGAAGGTGTCTTACTCAGAAAAAAAAATGCCTGGTCAGCAA
TTCACTGTCATGCACCTATTTTTAGATTGGGCAGGGAGATGGGAGGAGTCGTGATTTTTACTTTGAATAC
TATTTCACTGACGTTACTAAGCATTGCAGCACAATGTAGAAATTGGCTTGGGATGGATAGGTATAGGGAA
GGGCATTCGTTGGGAAATTGATGACCGGAAACTAGAAAGCCCCTATAATGTGGAATATGCATATGTGGGG
TGAAACCGTATACTACTTTCACTGCCATGACACTAGGCAAAAAATATTTCCTGTTAAAAGAAAGAGAAAA
AGAGAGGGATATTGAAAAATATAAGGAGTTGAGTCGCTTTGTGGTATAGAAGGGAGTAAGGAATAAAAGA
TATAGAATAGTAATCTAGTTGAACTGTTCTGCACAAAACATGCTTCCTTTTCAAAGAATAGACTTCAAAA
GGGACGAACAAACATTAGGTCACCTTTTGTGTTTATAATGAATTGTTGACGTTTCCATGCTGTGGTCTCT
TGATATACGGAAGTAGACGTAACTTATTTTCCAGCGAAATTATATACTTCTTCTGGGCTCCTTTGCTCAG
TTGCCCCAGGAAGGCTTCCCTGTGAGTAATGCTTATGGAATCTGACAAATGTGAATGAGCTGCCATTGGT
GGGAATCAGTAAACTTTGAAAACCTTTCACTGATGATTCCCTGGTAAGTGCTCCTGTGAGGGGCCACTTC
CCTAGTATGTGAGAGAGACCCTCTGCAAGCCGCTGTGGGGACTTAGCTTCCTGATACTAGGTAGTGATGG
TGGATCTAGAGACATTCACTTGAAGAGCAAGGATTGGGGTGGGGTGGGTTGGTGAATCTATCTCAGGCCA
CTGCACTGCAAGGTGCATTTCTGTCATTTCTGCAACACCATGAGACCTGAGCAAGAATGTGCTGTGGCAG
AGGGGGTCTTGGTTTCATAAATGGGTATATACAGCCTGAACTCAGGAGGCAGAAATCAAAAGAAATCCTT
GAGGGCTGGAACCTGAGGATAGAGCGAAAATGGAGAGAAGGGTGTTCAAGGTCAGGAGTGATTTTTGCAT
TTGGTGGAAAACCTCAAGTCAGACTCAGTGGCAGAACATGCAGCAGAAAAGCTGTGAAGGAAGGAAATTT
ACGTGATTGTAATGGAGGCTAGGGGTGGATTGGGAGGTGAGTGGAGGAGAGTCTCTGACCAGCTGCAGAG
CCAACCCCTATCATTTTATTAACATGGAACCCACACAGATAGGGGAGGGATTAAGCTAAGAACTTACAGA
ATGCAAACACGAGCACACTCTCTTCGAACCCAATTGTGGGTGTAGCAATGAAAGCAATATGATATGCTGC
AGTGTGAAGCTCCTTCTGGGGTTATCGTATGTACAAAGTTTACCTTATAATGGCTCAAATTGTATTTAAT
TTTTTTGTTTTGTTTATAAATGAAGAAAAGCTATAAGTATAATGTAATTATTTTATAGGTATACTATTAA
ATTATAGAACAAATAAAGATAATCTAGGATTATATGTGATCCCGGTGTGACGTGCCATAAAAGTGGAATT
TTTGACTTGTTGCAGTGAGCTGTGAAGCAGCACTATGCGTTCAGAGAGTTTTTGTTCAACCATGGTGAGT
TCAACCTCTAGCTTATGTCACATGATCTCCTTTGGGGTCTGGAATCTTCTAAGAGAAATAGAATCAGGCA
TCCAAGGAGCAGAAGTGATGGAGGTGGCCCTCAATCCTGGTCTTTGGAAGCTTCCAAGCCTCTTCCACAT
CCCCTGCCAGCTGAAGACAGAGTATTCTCACTGTGCTGGCTGAAGGAGAAACACTAAACAAGGAAACCTT
GAGAAACTGCAGCAATGGAAGTCCACTGTTAGTCTCTCCGTTAGCACCAGGGACACACTTGTTCTGAGTT
TTGTTCATTTTCACATTTACTCCTCATGAGTTCCAACATTTCTTAATTTAAATATTTCTTAGTTTATAGC
ATCTCCATTTTTCTATTAATTGACTTAATCTGCCATTTAAGAAAACTCCGTAATCAATAAAAAATAAAAT
CCACAATCAAACTGCACATGGTTGATCCACTGTGTAGGCTTCAAAGGAATTAAACTCAACTTCCTAGGAA
ATAATTCATCTTAGTTTATTAAATGGGACAGAATATTTCACTGCTAACGTATTCTAACTCACTTGTTTTT
GAAATTGGCCAGATGATTTTTAAGTTATAAAGTCAATGAAAATGACTTTTGCTATTGTGTGAACAACTTA
GAATGTATATATTCCCAGAAGGCTTATTTAATGATTGACCTATCACCACTGATTATTAATGTCTGCATGA
ATGATAACAATAAGACCAAGTCATCAATAATGCAATGAAAATTTTTTAAGTGAAAGCAAGTTTATTAAGA
AAGTAAAGGAATAAAGAATGCCTACTTCATAGGCAGAGCAGCCTAA

The term “isolated nucleic acid” refers to a nucleic acid that is separated from other nucleic acid molecules that are present in a mammalian genome, including nucleic acids that normally flank one or both sides of the nucleic acid in a mammalian genome. An isolated nucleic acid can be, for example, a DNA molecule, provided one of the nucleic acid sequences normally found immediately flanking that DNA molecule in a naturally occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

In some forms, the nucleic acids encoding GREM2 proteins include nucleic acids variants of SEQ ID NO:3. Variant nucleotide sequences include sequences that differ by one or more nucleotide substitutions, additions, or deletions, such as allelic variants. In some forms, the nucleic acid from about 70% to about 95% identical to SEQ ID NO:3. In some forms, the nucleic acid is at least 70%, 80%, 85%, 90%, 95% identical to SEQ ID NO:3. An isolated nucleic acid sequence or a recombinant nucleic acid sequence that is 97%, 98%, 99%, or 100% identical is also contemplated.

In some forms, the isolated nucleic acids that differ from the SEQ ID NO:3 due to the degeneracy of the genetic code are also described herein. For example, many amino acids are specified by more than one triplet. Codons that specify the same amino acid, or synonymous codons (e.g., CAU and CAC are synonymous codons for histidine) can result in “silent” mutations that do not affect the amino acid sequence of the protein. However, DNA sequence polymorphisms that cause changes in the amino acid sequence of the protein are believed to exist between mammalian cells. Those skilled in the art will recognize that these variations in one or more nucleotides (up to about 3% to 5% of the nucleotides) of the nucleic acid encoding a particular protein are due to natural allelic variations. Any and all such nucleotide variations and resulting amino acid polymorphisms are also contemplated.

Nucleic acids can be in sense or antisense orientation, or can be complementary to a reference sequence encoding a GREM2 polypeptide or protein. Thus, nucleic acids encoding SEQ ID NO:1 and SEQ ID NO:2, and fragments and variants thereof, in sense and antisense, and in single stranded and double stranded forms, are provided. The nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone. In preferred embodiments, the nucleic acid molecule is a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo.

### C. Vectors Expressing or Encoding GREM2 Proteins

In some embodiments, nucleic acids encoding GREM2 proteins are present within vectors. Nucleic acids in vectors can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Hamann, et al., J. Biol. Eng., 13:7 (2019) demonstrated that gene expression in hBMSCs driven by cytomegalovirus (CMV) promoter, resulted in 10-fold higher transgene expression than transfection with plasmids containing elongation factor 1 α (EF1α) or Rous sarcoma virus (RSV) promoters.

Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA). Recent transfection studies have investigated minicircle DNA (mcDNA), nucleic acids that are derived from pDNA by recombination that removes bacterial sequences. L1 RNA can be introduced into host cells using mcDNA using methods known in the art (Mun et al. Biomaterials, 2016; 101:310-320).

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### D. Pharmaceutical Formulations

The GREM2 compositions described herein can be formulated for administration to a subject in need thereof. Generally, the formulations are suitable for directly or indirectly increasing bone mineral density and reducing bone marker turnover in the cell, by at least 5%, 10%, 15%, 20%, 30%, 40, 50, 60, 70%, 80%, 90%, 95%, 100%. In some forms, the formulations are also suitable for directly or indirectly reducing the activity of BMP2 in the cell, by at least 20%, 30%, 40, 50, 60, 70%, 80%, 90%, 95%, 100%.

Exemplary formulations of GREM2 compositions include liquids and dry powders. In some forms, the GREM2 proteins, nucleic acids, or small molecules in an amount from about 1% to about 100%, inclusive, from about 1% to about 80%, from about 1% to about 50%, preferably from about 1% to about 40% by weight, more preferably from about 1% to about 20% by weight, most preferably from about 1% to about 10% by weight. The ranges above are inclusive of all values from 1% to 100%.

### 1. Parenteral Formulations

The compositions described herein (i.e., vectors encoding GREM2) can be formulated for parenteral administration. For example, parenteral administration may include administration to a patient intravenously, intradermally, intraperitoneally, intramuscularly, subcutaneously, by injection, by infusion, etc.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

### i. Controlled Release Formulations

The parenteral formulations described herein can be formulated for controlled release including immediate release, delayed release, extended release, pulsatile release, and combinations thereof.

### a. Nano- and Microparticles

For parenteral administration, the GREM2 compositions, and optional one or more additional active agents, can be incorporated into microparticles, nanoparticles, or combinations thereof that provide controlled release of the compounds and/or one or more additional active agents. In embodiments wherein the formulations contain two or more agents, the agents can be formulated for the same type of controlled release (e.g., delayed, extended, immediate, or pulsatile) or the agents can be independently formulated for different types of release (e.g., immediate and delayed, immediate and extended, delayed and extended, delayed and pulsatile, etc.).

For example, the GREM2 compositions and/or one or more additional active agents can be incorporated into polymeric microparticles, which provide controlled release of the drug(s). Release of the agent(s) is controlled by diffusion of the agent(s) out of the microparticles and/or degradation of the polymeric particles by hydrolysis and/or enzymatic degradation. Suitable polymers include ethylcellulose and other natural or synthetic cellulose derivatives.

Alternatively, the GREM2 compositions can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances, and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and triglycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300 °C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropyl methyl-cellulose, hydroxypropyl cellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of agent containing microparticles. Additionally, proteins, polysaccharides and combinations thereof, which are water-soluble, can be formulated with agent into microparticles and subsequently cross-linked to form an insoluble network. For example, cyclodextrins can be complexed with individual agent molecules and subsequently cross-linked.

### b. Method of Making Nano- and Microparticles

Encapsulation or incorporation of GREM2 compositions into carrier materials to produce agent-containing microparticles can be achieved through known pharmaceutical formulation techniques. In the case of formulation in fats, waxes or wax-like materials, the carrier material is typically heated above its melting temperature and the agent is added to form a mixture comprising agent particles suspended in the carrier material, agent dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, agent is added, and the molten wax-agent mixture is congealed under constant stirring as the mixture cools. Alternatively, the molten wax-agent mixture can be extruded and spheronized to form pellets or beads. These processes are known in the art. For some carrier materials it may be desirable to use a solvent evaporation technique to produce agent-containing microparticles. In this case agent and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, agent in a particulate form is homogeneously dispersed in a water-insoluble or slowly water-soluble material. To minimize the size of the agent particles within the composition, the agent powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture.

In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (glutaraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

### 2. Enteral Formulations

Suitable oral dosage forms include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Oral mRNA delivery using capsules is described in Abramson, et al., Matter, 5(3):975-987 (2022) (incorporated herein by reference), using branched hybrid poly(β-amino ester) mRNA nanoparticles.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropyl methylcellulose, hydroxypropyl cellulose, ethylcellulose, and veegum, and synthetic polymers such as acrylic acid and methacrylic acid copolymers, methacrylic acid copolymers, methyl methacrylate copolymers, amino alkyl methacrylate copolymers, polyacrylic acid/Poly methacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the agent and a controlled release polymer or matrix. Alternatively, the agent particles can be coated with one or more controlled release coatings prior to incorporation into the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended-release coatings. The coating or coatings may also contain the compounds and/or additional active agents.

### III. METHODS OF TREATMENT

Methods of using the disclosed compositions and formulations including, but not limited to, GREM2 proteins, nucleic acids and small molecules are provided.

Methods of treatment including cells and other therapeutic agents including GREM2 peptides, nucleic acids, and/or small molecules are described.

An exemplary method involves treating a subject (e.g., a human) having a disease, disorder, or condition by administering to the subject an effective amount of a pharmaceutical composition including genetically modified GREM2 peptides, nucleic acids, and/or small molecules. In some embodiments, the methods administer a pharmaceutical composition to express recombinant GREM2 proteins to a subject (e.g., a human) having a disease, disorder, or condition in an amount effective to treat the disease, disorder, or condition. For example, in some embodiments, the methods treat a disease or disorder associated with bone loss by administering to the subject, an effective amount of a pharmaceutical composition including recombinant GREM2 peptides and/or proteins. In another exemplary form, the methods treat a disease or disorder associated with an elevated expression of BMP, by administering to the subject, an effective amount of a pharmaceutical composition including recombinant GREM2 peptides and/or proteins.

In some forms, in vivo gene therapy can be employed, whereby genetic material encoding GREM2 peptides and/or molecules capable of upregulating GREM2, is transferred directly into the patient.

In these embodiments (i.e., in vivo gene therapy), genetic material is introduced into a patient by a virally derived vector or by non-viral techniques. In vivo nucleic acid therapy can be accomplished by direct transfer of a functionally active DNA into mammalian somatic tissue or organ in vivo. Nucleic acids be administered in vivo by viral means. A therapeutic gene expression cassette is typically composed of a promoter that drives gene transcription, the transgene of interest, and a termination signal to end gene transcription. Such an expression cassette can be embedded in a plasmid (circularized, double-stranded DNA molecule) as delivery vehicle. Plasmid DNA (pDNA) can be directly injected in vivo by a variety of injection techniques, among which hydrodynamic injection achieves the highest gene transfer efficiency in major organs by quickly injecting a large volume of pDNA solution and temporarily inducing pores in cell membrane. To help negatively charged pDNA molecules penetrate the hydrophobic cell membranes, chemicals including cationic lipids and cationic polymers have been used to condense pDNA into lipoplexes and polyplexes, respectively.

GREM2 peptides or nucleic acid molecules encoding GREM2 may be packaged into retrovirus vectors using packaging cell lines that produce replication-defective retroviruses, as is well-known in the art. Other virus vectors may also be used, including recombinant adenoviruses and vaccinia virus, which can be rendered non-replicating. Nucleic acids may also be delivered by other carriers, including liposomes, polymeric micro- and nanoparticles and polycations such as asialoglycoprotein/polylysine. Various techniques and methods for in vivo gene delivery using the disclosed vectors and carriers are known in the art (reviewed in Wang, et al., Discov. Med., 18(97):67-77 (2014). A major advancement in DNA vector design is minicircle DNA (mcDNA), which differs from pDNA in the lack of bacteria-derived, CpG-rich backbone sequences. When administered in vivo, mcDNA mediates safer, higher and more sustainable transgene expression than conventional pDNA.

### A. Effective Amounts

Formulations containing one or more GREM2 protein (s) or nucleic acid (s), and one or more therapeutic, prophylactic, and/or diagnostic agents typically include an effective amount of an admixture of the one or more GREM2 protein (s) or nucleic acid (s), and one or more therapeutic, prophylactic, and/or diagnostic agents. Effective amounts of the combined GREM2 protein (s) or nucleic acid (s) are provided herein. It will be appreciated that in some forms the effective amount of the one or more GREM2 protein (s) or nucleic acid (s) and one or more therapeutic, prophylactic, and/or diagnostic agents is different from the amount that would be effective for the one or more therapeutic, prophylactic, and/or diagnostic agents to achieve the same result when administered in the absence of the GREM2 protein (s) or nucleic acid (s).

When used for treating bone loss in a subject, the amount of GREM2 protein (s) or nucleic acid (s) present in a pharmaceutical dosage unit, or otherwise administered to a subject, can be the amount effective to reduce the production, inhibit the activation, or inhibit a signaling of Bone morphogenetic protein-2 (BMP2). In some forms, the compositions and formulations are suitable for directly or indirectly reducing the activity of BMP2 in the cell, by at least 20%, 30%, 40, 50, 60, 70%, 80%, 90%, 95%, 100%. In some forms, the amount of GREM2 protein (s) or nucleic acid (s) present in a pharmaceutical dosage unit, or otherwise administered to a subject, can be the amount effective to increase bone mineral density, and/or bone strength. Preferably, the amount of GREM2 protein (s) or nucleic acid (s) present in the pharmaceutical dosage unit, is administered to the subject in an amount effective to increase bone mineral density by 5 to 15% one year following administration.

In some forms, the amount of GREM2 protein (s) or nucleic acid (s) present in a pharmaceutical dosage unit, or otherwise administered to a subject, can be the amount effective to reduce the expression of one or more bone turnover markers. Bone turnover biomarkers (BTMs) are byproducts produced from the bone remodeling process that can be measured in urine or serum and are indicative of the rate of bone turnover. In some forms, the bone turnover marker is a bone resorption marker. Non-limiting examples of bone resorption markers include N-terminal telopeptide of type I collagen (NTX), C-terminal telopeptide of type I collagen (CTX), hydroxyproline, total pyridinoline, free deoxypyridinoline (DPD), Bone sialoprotein (BSP), and tartrate-resistant acid phosphatase 5b. In some forms, the bone turnover marker is a bone formation marker. Examples of bone formation markers include but are not limited to total alkaline phosphatase, bone-specific alkaline phosphatase, osteocalcin, and serum type 1 procollagen (C-terminal/N-terminal): C1NP or P1NP.

In some forms, the pharmaceutical composition containing the GREM2 protein (s) or nucleic acid (s) is administered to a subject in need thereof, to deliver the GREM2 protein (s) or nucleic acid (s) in an amount between about 0.1 mg and about 1,000 mg, inclusive, preferably between about 0.5 mg and about 100 mg, inclusive, more preferably between about 1 mg and about 15 mg, inclusive, for example, 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, and/or 15 mg. The dosage can be between about 0.01 mg/kg and about 100 mg/kg, inclusive, preferably between about 0.05 mg/kg and about 10.0 mg/kg, inclusive, more preferably between about 0.1 mg/kg and about 1.5 mg/kg, inclusive, for example, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, and/or 1.5 mg/kg.

### B. Dosage Regimens

A dosage regimen of the pharmaceutical composition containing the one or more GREM2 protein (s) or nucleic acid (s) and optionally one or more therapeutic, prophylactic, and/or diagnostic agents, can include one or multiple administrations of the pharmaceutical composition.

In some forms, the pharmaceutical compositions are administered at least 1, 2, 3, 4, 5, 6, 7, 8, 9, 10, 11, 12, 13, 14, 15, 16, 17, 18, 19, 20, 21, 22, 23, 24 or 30 hours, or more than 30 hours, up to 36 or 48 hours prior to or after the detection of the disease or symptoms associated with the disease in the patient. In other forms, the pharmaceutical compositions are administered at least 1, 2, 3, 4, 5, 6, 7, 8, 9, 10, 11, 12, 13, 14, 15, 16, 17, 18, 19, 20, 21, 22, 23, 24 or 30 hours, or more than 30 hours, up to 36 or 48 hours prior to or after administering a separate therapeutic, prophylactic, or diagnostic agent. In certain forms, additive or more than additive effects of the administration of the pharmaceutical composition containing the one or more GREM2 protein (s) or nucleic acid (s) in combination with one or more therapeutic and/or prophylactic agent (s) is evident after one day, two days, three days, four days, five days, six days, one week, two weeks, three weeks, or more than three weeks following administration.

An effective amount of the pharmaceutical compositions and optionally one or more therapeutic and/or prophylactic agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated. Such unit doses may be administered at other intervals, such as twice a week, thrice a week, weekly, bi-weekly, or monthly for up to six months or a year.

In some forms, the pharmaceutical compositions are administered to a subject in need thereof, for an unlimited time period. For example, a subject suffering from a disorder or disease associated with bone loss e.g., osteoporosis, may require treatment for an indefinite time period to prevent, delay, or retard the onset and/or to reduce the severity of symptoms associated with the disorder or disease. In these forms, the pharmaceutical composition can be administered for time periods including 1, 2, 3, 4, 5, 6, 7, 8, 9, 10, 11, 12 months, up to and including the entire lifetime of the subject being treated.

### C. Diseases to be Treated

Methods of treating diseases and/or disorders in a subject in need thereof are provided. The subject to be treated can have a disease, disorder, or condition such as but not limited to pre-osteoporosis, osteoporosis, osteopenia, bone fractures, and bone cancer. The disclosed compositions and methods can also be used to treat bone loss associated with various diseases. Such diseases include, for example, Rheumatoid Arthritis, Diabetes Mellitus type 1, Hyperthyroidism, Hyperparathyroidism, Celiac disease, Inflammatory bowel diseases such as Crohn's disease and ulcerative colitis.

“Osteoporosis” is defined in the art as a systemic skeletal disease characterized by low bone mass and microarchitectural deterioration of bone tissue, with a consequent increase in bone fragility and susceptibility to fracture. Any bone can be affected by osteoporosis, although the hip, spine, and wrist are common bones that are broken or fractured in subjects suffering from or at risk for osteoporosis. Osteoporosis in is defined as a value for bone mineral density (BMD) of >2.5 SD below the young average value, i.e., a T-score of −2.5 SD. Severe osteoporosis (established osteoporosis) uses the same threshold, but with one or more prior fragility fractures. The preferred site for diagnostic purposes is BMD measurements made at the hip, either at the total hip or the femoral neck. For men, the same threshold as utilized for women is appropriate, since for any given BMD, the age adjusted fracture risk is more or less the same.

“Osteopenia” is a pre-osteoporosis condition characterized as a mild thinning of bone mass which is not as severe as osteoporosis. Osteopenia results when the formation of bone is not enough to offset normal bone loss. Osteopenia is generally considered the first step along the road to osteoporosis, defined by a value of bone mineral density (BMD) of between 1SD and 2.5SD below the young average value, i.e., a T-score of −1SD to −2.5SD. Diminished bone calcification can also be referred to as osteopenia, whether or not osteoporosis is present.

“Pre-Osteoporosis” encompasses both osteopenia and also other conditions which result in a high risk of future development of osteopenia, osteoporosis, and bone fracture. Subjects who are deemed clinically to be at low risk or no risk for developing osteoporosis or osteopenia based on current BMD nevertheless may still be at risk for pre-osteoporosis or bone fracture, as BMD measures bone status at the time of assessment and not rate of bone metabolism or predisposition to a lowered future BMD. The majority of bone fractures occur in subjects who have not been previously diagnosed with osteoporosis or pre-osteoporosis. There is a substantial need for better risk assessment and stratification tools for those who do not yet have osteoporosis or osteopenia yet are expected to have higher than normal rates of progression to those symptomatic disease states measurable by BMD.

In some forms, the methods include administering the GREM2 compositions to a subject in need, for the treatment of fractures and cartilage defects, e.g., in humans and other animals. In some forms, the methods include administering the GREM2 compositions to a subject in need for prophylactic use in reducing closed and open fractures, and also in improving prosthesis consolidation. New bone formation induced by osteogenic agents contributes to the repair of craniofacial defects congenital, traumatically induced or induced by tumor resection and is also useful for cosmetic surgery. In certain cases, the GREM2 compositions may provide an environment that attracts osteogenic cells, may stimulate the growth of osteogenic cells, and of osteogenic cell precursor cells. It may also induce differentiation.

In some forms, the methods include administering the GREM2 compositions to a subject in need, for the treatment of bone loss associated a disease or disorder including but not limited to hyperparathyroidism-, chronic kidney disease e.g., bone mineral disorder, sex hormone deprivation or sex hormone ablation (Androgen and/or estrogen), rheumatoid arthritis, severe burn injury e.g., bone resorption and low bone formation, hyperparathyroidism, hypercalcemia, hypocalcemia, hypophosphatemia, osteomalacia (tumor-induced), Hyperphosphatemia, vitamin D deficiency, hyperparathyroidism (including familial hyperparathyroidism) and pseudohypoparathyroidism, tumor metastasis to bone. In some forms, the methods include administering the GREM2 compositions to a subject in need, for the treatment of bone loss associated a disease or disorder including but not limited to bone loss as a result of tumor or chemotherapy, e.g., bone and bone marrow tumors, Ischemic bone disorder, periodontal disease and oral bone loss, Cushing disease, Paget's disease, thyroid poisoning, chronic diarrhea or malabsorption, tubular acidosis, or anorexia nervosa.

Particular conditions which can be treated using the disclosed compositions and methods include dysplasias, wherein growth or development of bone is abnormal and a wide variety of causes of osteopenia, osteoporosis and bone loss. Representative examples of such conditions include achondroplasia, cleidocranial dysostosis, enchondromatosis, fibrous dysplasia, Gaucher's Disease, hypophosphatemia rickets, Marfan's syndrome, multiple hereditary exostoses, neurofibromatosis, osteogenesis imperfecta, osteopetrosis, osteopoikilosis, sclerotic lesions, pseudoarthrosis, and pyogenic osteomyelitis, periodontal disease, anti-epileptic drug induced bone loss, primary and secondary hyperparathyroidism, familial hyperparathyroidism syndromes, weightlessness induced bone loss, osteoporosis in men, postmenopausal bone loss, osteoarthritis, renal osteodystrophy, infiltrative disorders of bone, oral bone loss, osteonecrosis of the jaw, juvenile Paget's disease, melorheostosis, metabolic bone diseases, mastocytosis, sickle cell anemia/disease, organ transplant related bone loss, kidney transplant related bone loss, systemic lupus erythematosus, ankylosing spondylitis, epilepsy, juvenile arthritis, thalassemia, mucopolysaccharidoses, Fabry disease, turner syndrome, Down Syndrome, Klinefelter Syndrome, leprosy, Perthes' Disease, adolescent idiopathic scoliosis, infantile onset multi-system inflammatory disease, Winchester Syndrome, Menkes Disease, Wilson's Disease, ischemic bone disease (such as Legg-Calve-Perthes disease, regional migratory osteoporosis), anemic states, conditions caused by steroids, glucocorticoid-induced bone loss, heparin-induced bone loss, bone marrow disorders, scurvy, malnutrition, calcium deficiency, idiopathic osteopenia or osteoporosis, congenital osteopenia or osteoporosis, alcoholism, chronic liver disease, postmenopausal state, chronic inflammatory conditions, rheumatoid arthritis, inflammatory bowel disease, ulcerative colitis, inflammatory colitis, Crohn's disease, oligomenorrhea, amenorrhea, pregnancy, diabetes mellitus, hyperthyroidism, thyroid disorders, parathyroid disorders, Cushing's disease, acromegaly, hypogonadism, immobilization or disuse, reflex sympathetic dystrophy syndrome, regional osteoporosis, osteomalacia, bone loss associated with joint replacement, HIV associated bone loss, bone loss associated with loss of growth hormone, bone loss associated with cystic fibrosis, fibrous dysplasia, chemotherapy associated bone loss, tumor induced bone loss, cancer-related bone loss, hormone ablative bone loss, multiple myeloma, drug-induced bone loss, anorexia nervosa, disease associated facial bone loss, disease associated cranial bone loss, disease associated bone loss of the jaw, disease associated bone loss of the skull, and bone loss associated with space travel. Further conditions relate to bone loss associated with aging, including facial bone loss associated with aging, cranial bone loss associated with aging, jawbone loss associated with aging, and skull bone loss associated with aging.

In some forms, the methods include administering the GREM2 compositions to a subject in need, for the treatment of non-adhesive fractures, fractures that are slow to heal, fetal and neonatal dysplasia (e.g., hypocalcemia, hypercalcemia, calcium receptor deficiency, and vitamins). It can also be applied to conditions characterized by bone formation or healing failure, including D deficiency), osteonecrosis (including jaw osteonecrosis), and osteogenesis imperfecta.

In some forms, the GREM2 compositions and formulations are administered to a subject in need to reduce one or more symptoms associated with bone loss, e.g., decreased bone mineral density, decreased bone strength, decreased bone volume/tibial volume ratio (BV/TV), and increased fracture risk. Other than direct measurement of BMD, several conventional risk factors for osteoporosis and bone fracture are often assessed prior to or in parallel with a diagnosis of osteoporosis or assessment of pre-osteoporosis conditions. Such risk factors include, without limitation, gender, wherein the chances of developing osteoporosis or osteopenia are greater in females due to less bone tissue as well as changes that happen during menopause; age, wherein bones become thinner and weaker with age; small body size; ethnicity, wherein Caucasian and Asian women are at highest risk and African American and Hispanic women have a lower but significant risk; family history, wherein fracture risk is thought to be due, in part, to genetics. Subjects whose parents have a history of fractures are reported to also have reduced bone mass and may be at risk for fractures.

Other significant risk factors include abnormally low levels of sex hormones, indicated by the abnormal absence of menstrual periods (amenorrhea), low estrogen levels such as found during female menopause (including, without limitation, low levels of any one or more of the primary estrogens, estradiol, estriol, and estrone, and their intermediates, precursor androgens and estrogen derivatives), and low testosterone level such as found in older men. Subjects suffering from anorexia nervosa are also at increased risk for osteoporosis. Diets low in calcium and vitamin D can also result in a higher incidence of bone loss. Subjects who undergo long-term use of glucocorticoids and some anticonvulsants can also lead to loss of bone density and fractures. Subjects who exhibit these risk factors frequently are found to have osteoporosis or a pre-osteoporosis condition when assessed by BMD. Also at risk for developing osteoporosis or osteopenia are subjects who lead inactive lifestyles or who have been subjected to extended bed rest, subjects who engage in smoking, or excessive consumption of alcohol. Several risk rules and indices have been constructed integrating these variables into clinically useful measurements of absolute or relative risk, such as the Osteoporosis Risk Assessment Instrument (ORAI), the Osteoporosis Self-Assessment Tool (OST), among others; such multi-variate approaches tend to have reasonably high sensitivity for osteoporosis, but low specificity. For example, the OST has been reported to identify over 90 percent of women with osteoporosis (and 100% of those over 65), but more than half of the women identified by this tool as requiring BMD resting were found on test to actually not have osteoporosis (See Chapter 10, Bone Health and Osteoporosis: A Report of the Surgeon General (2004) and also Woolf & Pfleger, Burden of Major Musculoskeletal Conditions, Bulletin of the World Health Organization (2003) 81: 646-656).

In some forms, the disease, disorder, or condition can be associated with an elevated expression or specific expression of a protein e.g., bone morphogenetic protein. For example, the GREM2 compositions can be administered to a subject in need to reduce the expression of one or more bone turnover markers. Bone turnover biomarkers (BTMs) are byproducts produced from the bone remodeling process that can be measured in urine or serum and are indicative of the rate of bone turnover. In some forms, the bone turnover marker is a bone resorption marker. Non-limiting examples of bone resorption markers include N-terminal telopeptide of type I collagen (NTX), C-terminal telopeptide of type I collagen (CTX), hydroxyproline, total pyridinoline, free deoxypyridinoline (DPD), Bone sialoprotein (BSP), and tartrate-resistant acid phosphatase 5b. In some forms, the bone turnover marker is a bone formation marker. Examples of bone formation markers include but are not limited to total alkaline phosphatase, bone-specific alkaline phosphatase, osteocalcin, and serum type 1 procollagen (C-terminal/N-terminal): C1NP or P1NP.

### D. Subjects to be Treated

A subject in need of treatment is a subject having a disease associated with bone loss or a subject expressing one or more symptoms associated with bone loss. Exemplary diseases associated with bone loss include osteopenia, osteoporosis, pre-osteoporosis, or bone cancer. In some forms, the subject is a mammal, including, but not limited to, murines simians, humans, mammalian farm animals and livestock, mammalian sport animals, and mammalian pets. Preferably, the subject is a human.

Methods of assessing and detecting bone loss in a subject are known by those of ordinary skill in the art. An apparently healthy subject is a subject who has no signs or symptoms of disease.

The effect of the pharmaceutical compositions including one or more GREM2 protein (s) or nucleic acid (s) can be compared to a control. Suitable controls are known in the art and include, for example, an untreated subject, or a placebo-treated subject. A typical control is a comparison of a condition or symptom of a subject prior to and after administration of the pharmaceutical compositions including one or more GREM2 protein (s) or nucleic acid (s). The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some forms, the symptom, pharmacologic, or physiologic indicator is measured in a subject prior to treatment, and again one or more times after treatment is initiated. In some forms, the control is a reference level, or average determined based on measuring the symptom, pharmacologic, or physiologic indicator in one or more subjects that do not have the disease or condition to be treated (e.g., healthy subjects). In some forms, the effect of the treatment is compared to a conventional treatment that is known the art. Suitable control subjects are unvaccinated subjects, or subjects receiving the same amount of a therapeutic, prophylactic and/or diagnostic agent in the absence of pharmaceutical compositions including one or more GREM2 protein(s) or nucleic acid(s).

### E. Routes of Administration

The pharmaceutical compositions containing one or more GREM2 protein (s) or nucleic acid (s) are generally administered to a subject in an effective amount. As used herein the term “effective amount” means a dosage sufficient to inhibit, or prevent one or more infections, or symptoms of a disease or to otherwise provide a desired pharmacologic and/or physiologic effect. The precise dosage will vary according to a variety of factors such as subject-dependent variables (e.g., age, immune system health, etc.), the specific stage of disease, and the treatment being affected.

In some forms, the disclosed pharmaceutical composition containing GREM2 peptides and/or small molecules are administered parenterally. The phrases “parenteral administration” and “administered parenterally” are art-recognized terms, and include modes of administration other than enteral and topical administration, such as injections, and include without limitation intravenous, intramuscular, intrapleural, intravascular, intrapericardial, intraarterial, intrathecal, intratracheal, intranasal intracapsular, intraorbital, intracardiac, intradennal, intraperitoneal, transtracheal, subcutaneous, subcuticular, intraarticular, subcapsular, subarachnoid, intraspinal and intrastemal injection and infusion. The disclosed pharmaceutical composition containing one or more GREM2 protein (s) or nucleic acid (s) can be administered parenterally, for example, by subdural, intramuscular, intravenous, intrathecal, intraventricular, intraarterial, intra-amniotic, intraperitoneal, or subcutaneous routes. In preferred forms, the disclosed pharmaceutical composition containing one or more GREM2 protein (s) or nucleic acid (s) are administered via intraperitoneal, intravenous, intramuscular, subcutaneous, and intradermal administration. The most preferred forms of administration are subcutaneous, intravenous, and intramuscular.

### IV. KITS

Medical kits are also disclosed. The medical kits can include, for example, a dosage supply of pharmaceutical composition containing one or more GREM2 peptides and/or small molecules, and optionally one or more therapeutic, prophylactic or diagnostic agents, either separately or together in the same admixture. The active agents can be supplied alone (e.g., lyophilized), or in a pharmaceutical composition. The active agents can be in a unit dosage, or in a stock that should be diluted prior to administration. In some forms, the kit includes a supply of pharmaceutically acceptable carrier. The kit can also include devices for administration of the active agents or compositions, for example, syringes. The kits can include printed instructions for administering the compound in a use as described above.

The disclosed compositions and methods can be further understood through the following numbered paragraphs.

1. A method of treating a subject having a disease, disorder, or condition associated with bone loss, the method comprising administering to the subject a composition comprising:
(a) an effective amount of a soluble protein comprising all or a functional portion of amino acids 22 to 168 of a Gremlin-2 (GREM2) protein, or a functional variant thereof, wherein the function of the functional portion and functional variant comprises all or at least 20% of the native GREM2 protein antagonism to Bone Morphogenic Protein-2 (BMP2); or
(b) a nucleic acid encoding the soluble protein of (a), wherein the nucleic acid includes sequences mediating expression of the soluble protein in cells of the subject after the administration.

2. The method of paragraph 1, wherein the soluble protein comprises the amino acid sequence SEQ ID NO:2, or a functional variant thereof having an amino acid sequence at least 70%, 75%, 80%, 85%, 90%, 95%, or 99% identical to any one of SEQ ID NO:2.

3. The method of paragraphs 1 or 2, wherein the amino acid sequence of the soluble protein comprises at least 70% and less than 100% sequence identity to any one of SEQ ID NO:2.

4. The method of any one of paragraphs 1-3, wherein the functional variant binds to BMP2 with at least 60% of the affinity of GREM2 protein.

5. The method of any one of paragraphs 1-4, wherein the functional variant includes the sequence CX₆QX₆CX₆NX₂CXGXCXSX₃PX₍₈₋₁₃₎CX₂CXPX₈TLXCX₍₁₅₋₁₈₎ CXC (SEQ ID NO: 4).

6. The method of any one of paragraphs 1-5, wherein the nucleic acid is RNA or DNA.

7. The method of paragraph 6, wherein the nucleic acid is modified or unmodified oligonucleotide; selected from the group comprising antisense oligonucleotides, short interfering RNA, microRNA, peptide nucleic acids, locked nucleic acids, aptamers, and spiegelmers.

8. The method of any one of paragraphs 1-7, wherein the nucleic acid is mRNA.

9. The method of any one of paragraphs 1-8, wherein the nucleic acid comprises one or more expression control sequence(s).

10. The method of any one of paragraphs 1-9, wherein the nucleic acid is, or is encoded by, a vector or a transposon.

11. The method of paragraph 10, wherein the vector is a viral vector.

12. The method of paragraph 11, wherein the viral vector is selected from the group consisting of a lentiviral vector, an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector comprising a combination of any two or more of an Adeno-associated virus (AAV) vector, Herpes Simplex virus (HSV) vector, vesicular stomatitis (VSV) vector, or a human Bocavirus vector (hBoV).

13. The method of paragraph 10, wherein the vector is a nucleic acid expression vector selected from the group consisting of a plasmid, a cosmid, and a replicon.

14. The method of any one of paragraphs 1-13, wherein the composition is administered at an interval selected from the group consisting of once a day, twice a day, once a week, twice a week, three times a week, four times a week, once every two weeks, once every three weeks, approximately once a month, once every two months, once every three months, once every four months, once every six months, once every eight months, once every nine months, and once a year.

15. The method of any one of paragraphs 1-14, wherein the composition is administered once or more times a week for up to a period of 1, 2, 3, 4, 5, 6, 7, 8, 9, 10, 11, 12 months, or more.

16. The method of any one of paragraphs 1-15, wherein the subject is human.

17. The method of any one of paragraphs 1-16, wherein the composition is administered to a human subject at a dose of between 0.01 mg/kg body weight of the subject and 100 mg/kg body weight of the subject, inclusive.

18. The method of any one of paragraphs 1-17, wherein the composition is administered in an amount effective to reduce the expression of bone-morphogenetic protein (BMP) in the subject.

19. The method of any one of paragraphs 1-18, wherein the composition is administered in an amount effective to reduce one or more bone turnover markers in a subject.

20. The method of any one of paragraphs 1-19, wherein the disease or disorder to be treated is selected from the group comprising pre-osteoporosis, osteoporosis, osteopenia, bone fractures, and/or bone cancer.

21. The method of any one of paragraphs 1-20, wherein the composition is administered orally, by injection, or by infusion.

22. The method of paragraph 21, comprising subcutaneously, intravenously, intramuscularly, intraperitoneally, or intradermally administering the composition to the subject.

23. The method of any one of paragraphs 1-22, wherein the composition further comprises a pharmaceutically acceptable buffer, carrier, diluent, or excipient.

The present invention will be further understood by reference to the following non-limiting examples.

### Examples

### Materials and Methods

### Transcriptome Wide Association Study (TWAS), Colocalization Analysis and Summary Data Based Mendelian Randomization Analysis.

Using publicly available data, the association of GREM2 gene expression with BMD was evaluated. A TWAS was conducted using S-PrediXcan (Gamazon, E. R., et al., A gene-based association method for mapping traits using reference transcriptome data. Nature Genetics, 2015. 47(9): p. 1091-1098). Using expression quantitative trait loci (eQTL) from 49 different tissues of GTEx v7 (The Genotype-Tissue Expression (GTEx) project. Nat Genet, 2013. 45(6): p. 580-5) as exposure and heel estimated BMD [10] as the outcome, genes whose expression is associated with BMD were identified. To evaluate causality, a colocalization analysis was conducted (Giambartolomei, C., et al., Bayesian Test for Colocalisation between Pairs of Genetic Association Studies Using Summary Statistics. PLOS Genetics, 2014. 10(5): p. e1004383) and summary data based mendelian randomization analysis (SMR) (Zhu, M., et al., Integrating genome-wide association and transcriptome prediction model identifies novel target genes for osteoporosis. Osteoporosis International, 2021. 32(12): p. 2493-2503) on the resulting genes were generated. A causal association was considered if the significant p-value was significant in both S-PrediXcan and SMR analyses. Causal associations must also have insignificant associations for the posterior probability of colocalization (p_coloc) and the HEIDI test, which were the supplementary analyses of SMR.

### Trap Staining and Calcium Phosphate Resorption Assay.

Murine RAW 264.7 macrophage cells (ATCC TIB-71) were maintained in DMEM (ATCC 30-2002) supplemented with 10% FBS (Gibco) and Pen/Strep 100 unit/ml (Gibco). Cells were incubated at 37 °C in a 5% CO₂ atmosphere. RAW264.7 cells were cultured in 96-well plate (SPL Life Sciences #30096) for TRAP-staining and RNA extraction, respectively. DMEM supplemented with 10% FBS, and Pen/Strep was used as basal medium for RAW264.7 cell differentiation. To generate osteoclast, RAW 264.7 cell was seeded at 6000 cells/cm² for 24 hours. Medium was then replaced with 10 ng/ml RANKL (R&D system) with or without GREM2 protein (R&D system; #2069-PR-050/CF; recombinant mouse) to start differentiation. Medium was replaced every 3 days. Cell culture was harvested on day 5 and cells were fixed and stained for TRAP activity using TRAP staining kit (Sigma Aldrich #387A-1KT) according to the manufacturer's instructions. After drying the plate over-night at room temperature, TRAP-positive multinucleated (nuclei >3) cells were counted as osteoclast using a light microscope (Nikon Eclipse TS100).

Calcium phosphate surface resorption assay was performed on Osteo Assay Surface 96-well Plate (Corning #3988). To analyse the surface of pit formation, the media was aspirated from the wells on day 5, and 100 μL of 10% bleach solution was added. Cells were incubated with the bleach solution for 5 minutes at room temperature. The wells were washed twice with distilled water and allowed to dry at room temperature for overnight. Image of each well was recorded using stitching function of a color imaging microscope (Olympus DP74) at 100× magnification. Stitched microscope image was post-processed and analyzed by Adobe PhotoShop 2020 CC and resorption area was calculated using Image J software. The effect of GREM2 treatment on osteoclast resorption activity using in vitro pit formation assay was examined.

### Cross-Sectional Study in Humans

A cross-sectional study in 1104 subjects from the Hong Kong Osteoporosis Study (Cheung et. al. (2018). Serum GREM2 was categorized into sex-specific quartiles and association between quartiles of serum GREM2 and bone mineral density at the lumbar spine, total hip and femoral neck were evaluated using analysis of covariance (ANCOVA). The association between serum GREM2 and bone resorption markers (CTx and NTx) as well as bone formation markers (P1NP and Osteocalcin) were also evaluated. The association was adjusted for age, sex, height, weight, status of drinking, status of smoking and physical activity.

### Production of Recombinant Mouse GREM2

### 1. Expression Cell Stock

GREM2 expression plasmid was transfected into BL21(DE3) Competent Cells according to manufacturer's instructions. Single clones of E coli carrying GREM2 expression plasmid was selected by AMP agar plate and then grown overnight in LB+AMP+2% glucose at 37 °C, 220 rpm. Glycerol stocks of single clones were prepared by mixing 1:1 ratio of overnight culture and sterile 50% glycerol and stored at −80 °C.

### 2. Mouse GREM2 Inclusion Body Expression

Mouse GREM2 expression seed culture was prepared in 25 mL LB+ampicillin (100 μg/ml)+2% glucose from glycerol stock and shaken overnight at 37 °C, 220 rpm. On the other day, 24 mL of seed culture was added into 1 L of 2×YT medium with ampicillin (100 μg/ml) in baffled flask. Cell culture was induced at 0.8-1.0 OD with 0.5 mM IPTG for 4 or 6 hours. Cell pellet was harvested by 4696×g centrifugation for 15 minutes at 4 °C. Cell pellet was stored at −80 °C until further processing.

### 3. Purification of Inclusion Body

Two buffers were used for purification of inclusion body, Cell lysis buffer (50 mM Tris, 5 mM EDTA, pH 8.0), IB wash buffer (50 mM Tris, 5 mM EDTA, 0.5M NaCl, 0.5% Triton X-100, pH 8.0). Around 80 g of cell pellet was resuspended in 320 mL of cell lysis buffer (about 25% weight by wet cell) by sonication (40% power of 700 W) cycles on ice until homogenized. Inclusion body was collected by centrifugation at 5000×g for 30 minutes at 4 °C and supernatant was discarded. Inclusion body was then resuspended by sonication and washed 5 rounds by IB wash buffer and a final wash with Cell lysis buffer.

### 4. Mouse GREM2 Inclusion Body Solubilization and Clarification

About 6 g of wet IB was solubilized in 60 mL of denature buffer (100 mM Tris, 8M Urea, 1 mM EDTA, 10 mM DTT pH 8.5). It was rotated for 4 hours at room temperature. Solubilized GREM2 was then clarified by centrifuge 4696×g for 10 minutes at 4 °C and then supernatant was further centrifuged at 20000×g for 10 minutes at 4 °C.

### 5. Cation-Exchange Chromatography Purification of Denatured Mouse GREM2

50 mL of clarified denatured mouse GREM2 protein was loaded onto 5 mL HiTrap SP HP column (Cytiva #17115201) equilibrated with Buffer A (20 mM MES, 6M urea, pH 6.0). Column was washed by 25 ml of Buffer A and then eluted by linear 0-100% gradient buffer B (20 mM MES, 6M urea, 1M NaCl, pH 6.0). Protein was eluted in 50 mL volume with 5 ml fractions. Target protein bands purity was checked by SDS-PAGE and then selected for protein refolding.

### 6. Mouse GREM2 Protein Refolding

Denatured GREM2 protein was diluted in refolding buffer (50 mM Tris, 0.5M Arginine, 1 mM EDTA, 5 mM GSH, 1 mM GSSG, 0.5 mM cysteine, pH 8.5) at final concentration around 0.1 mg/ml drop by drop with stirring at room temperature. The refolding mixture was then incubated at room temperature for 3 days.

### 7. Cation-Exchange (CNX) Chromatography Purification of Refolded Mouse GREM2

Refolding mixture was diluted 2-fold by ultrapure water and then filtered by 0.2 μm bottle-top filter before loading onto 5 mL HiTrap SP HP column (Cytiva #17115201) equilibrated with Buffer A1 (0.25M Arginine, 50 mM MES, pH 6.0). Column was washed by 45 mL of Buffer A1 and then eluted by linear 0-100% gradient buffer B1 (1M Arginine, 50 mM MES, pH 6.0). Protein was eluted in 50 mL volume with 5 ml fractions. Target protein bands purity was checked by SDS-PAGE.

### 8. Dialysis of Purified Mouse GREM2

CNX chromatography purified protein with >95% purity was pooled together. The protein solution was then dialyzed by SnakeSkin Pleated Dialysis Tubing (7000 MWCO) three times in 50-volume of dialysis buffer (20 mM MES, pH 6.0) to remove arginine. Protein solution after dialysis was sterilized by filter through 0.2 μm syringe filter and stored at −20 °C.

### 9. SDS-PAGE Assay

Protein concentration of final GREM2 protein was estimated by BCA assay. Gel running sample was prepared by diluting GREM2 protein into final buffer MES buffer at pH 6.0 with 0.5M arginine and DTT reducing SDS-sample buffer. 5 μg of protein per lane was loaded into 18% SDS-PAGE gel and run for 6 hours at 100V. Unstained protein ladder was used as size reference.

### 10. BMP-2 Inhibition Dual Luciferase Activity QC Assay

BMP Responsive Reporter Osteoblast Cell Line (BRITER) (Cat #EBI001) was purchased from Kerafast Ltd and dual luciferase assay kit was purchased from Sigma-Aldrich (Cat #SCT152). BRITER cell line was cultured in DMEM high glucose with 10% FBS and 1% Pen/Strep (culture medium) and serum starved in DMEM high glucose with 0.3% FBS and 1% Pen/Strep (serum starvation medium).

BRITER cell was grown to confluence in 96-well plate. Culture medium was washed away by rinsing cells with 200 uL per well of 1×PBS once. And then cell was cultured with serum starvation medium for 2 hours at 37 °C, 5% CO₂. At around 1 hour incubation time, GREM2 protein at various concentration was started to pre-incubated with 1 nM of BMP-2 in serum starvation medium. The pre-incubation time is about 45 min to 1 hour before adding into serum starved BRITER cell line. BRITER cell line was then incubated with BMP-2 or GREM2/BMP-2 mixture for 2 hours. Finally, cell in 96 well plate was rinsed once by 1×PBS and lysed by 50 μL of passive lysis buffer per well by gentle pipetting. Luciferase assay was carried out according to the manufacturer's instruction.

### Treatment of GREM2 in Healthy Female C57BL/6J Mice.

10-week-old female C57BL/6J mice (Jackson Laboratory) were ordered and allowed to acclimatize for 2 weeks. A total of 12 mice were randomly allocated to either the treatment group (N=6) or control group (N=6). Recombinant mouse GREM2 protein (0.33 ug/g) was delivered into the body of mice in the treatment group by subcutaneous injection three times a week for 4 weeks (week 0 to week 4). Control group mice were similarly treated using the vehicle buffer instead. BMD and trabecular bone volume to total volume fraction (BV/TV) of mice were checked by micro-CT scanning under anaesthesia at week 0 and week 4. A one-tailed t-test was used to compare the mean BMD and BV/TV of GREM2 treated mice and the controls.

### Treatment of GREM2 in Ovariectomized (OVX) Female C57BL/6J Mice.

8-week-old female C57BL/6J mice (Jackson Laboratory) were ordered and allowed to acclimatize for 2 weeks. At age of 10-weeks, mice were ovariectomized to produce the OVX mice mimicking postmenopausal osteoporosis. Mice were allowed to recover for a period of 2 weeks. Then, a total of 12 mice were randomly allocated to either the treatment group (N=6) or control group (N=6). Recombinant mouse GREM2 protein (0.33 ug/g) was delivered into the body of mice in the treatment group by subcutaneous injection three times a week for 4 weeks (week 0 to week 4). Control group mice were similarly treated using the vehicle buffer instead. BMD and trabecular bone volume to total volume fraction (BV/TV) of mice were checked by micro-CT scanning under anaesthesia at week 0 and week 4. A one-tailed t-test was used to compare the mean BMD and BV/TV of GREM2 treated mice and the controls.

### Results

GREM2 Expression is Causally Associated with BMD.

Table 1 shows the results of the TWAS analysis. GREM2 expression was found to be significantly positively associated with BMD in S-PrediXcan and SMR. In addition, an insignificant association in coloc and HEIDI were observed. These results show that the higher gene expression of GREM2 is causally associated with BMD.

TABLE 1
Association between GREM2 Expression and BMD

| Gene | b_PrediXcan | p_PrediXcan | p_coloc | b_SMR multi | p_SMR_multi | p_HEIDI |
|---|---|---|---|---|---|---|
| GREM2 | 0.229 | 1.19E−141 | 1 | 0.212122 | 9.51E−10 | 0.082167 |

GREM2 is a negative regulator of osteoclastogenesis and bone resorption.

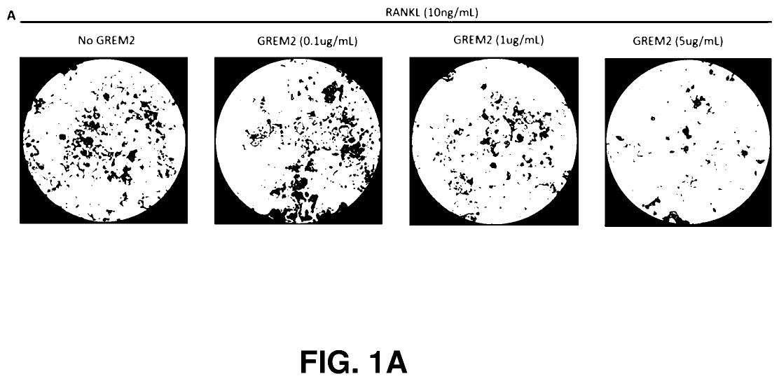

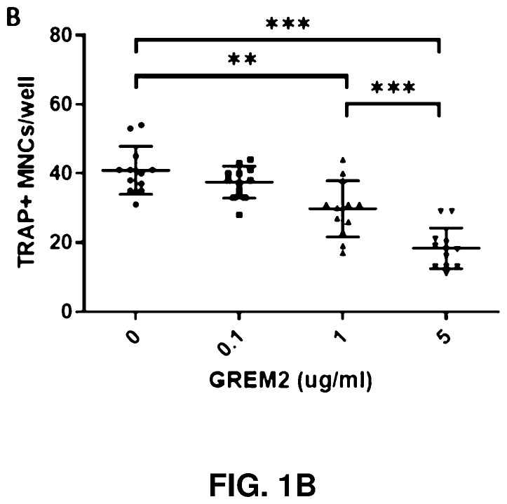

The TRAP-positive multinucleated osteoclasts were significantly reduced in a dose-dependent manner by GREM2 treatment (FIGS. 1A and 1B). In comparison with the control (RANKL-treated cells without GREM2 treatment), 27.3% and 55.2% reduction in number of osteoclasts was observed in cells treated with 1 and 5 μg/mL of GREM2, respectively.

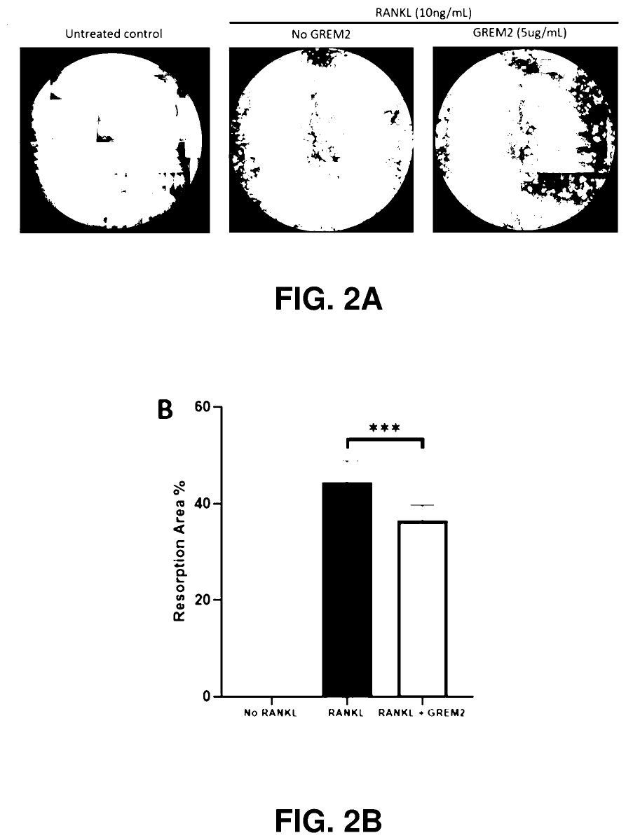

Resorption pits were formed on the calcium phosphate surface by osteoclasts after RANKL treatment. The percentage of resorption area was significantly reduced by 18.0% with 5 μg/mL of GREM2 treatment (FIGS. 2A and 2B).

Serum Level of GREM2 is Associated with Increased BMD and Reduced Levels of Bone Turnover Markers in Humans.

Using the lowest quartile as a reference, it was observed that the highest quartile of serum GREM2 was significantly associated with higher BMD at all sites measured and reduced bone formation (P1NP and OC) and resorption (CTx and NTx) markers (Table 2).

TABLE 2
Association between highest quartile of serum GREM2 and BMD or
Bone turnover markers using the lowest quartile as reference.

| | | | Lower | Upper | |
|---|---|---|---|---|---|
| Outcome | Estimate | S.E. | 95% CI | 95% CI | P Value |
| Lumbar Spine | 0.042 | 0.013 | 0.016 | 0.067 | 0.001* |
| Total Hip BMD | 0.022 | 0.01 | 0.003 | 0.041 | 0.020* |
| Femoral Neck BMD | 0.019 | 0.01 | 0.001 | 0.038 | 0.042* |
| P1NP | −0.191 | 0.094 | −0.375 | −0.007 | 0.042* |
| OC | −0.193 | 0.093 | −0.375 | −0.011 | 0.038* |
| CTx | −0.287 | 0.092 | −0.468 | −0.106 | 0.002* |
| NTx | −0.345 | 0.087 | −0.517 | −0.174 | <0.001* |

GREM2 Treatment in Healthy Mice Results in Increased BMD and BV/TV when Compared to Controls.

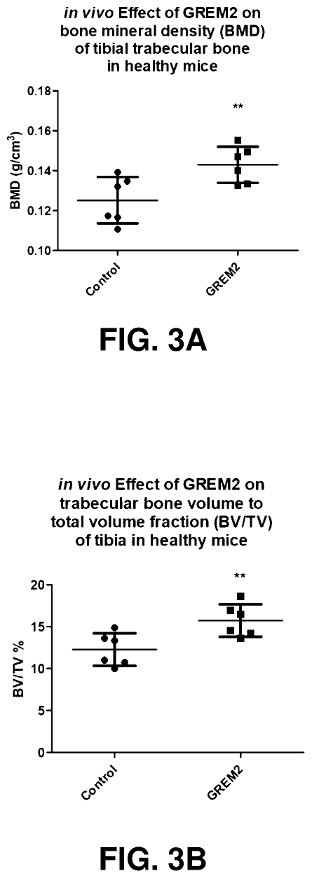

After treating the healthy mice with recombinant GREM2 for 4 weeks, it was found that, BMD and BV/TV at the proximal tibia was significantly higher in the treatment group when compared to the controls. The mean difference was −0.018 g/cm³ (p=0.007) for BMD and −3.466 (p=0.006) for BV/TV (FIGS. 3A and 3B).

GREM2 Treatment in OVX Mice Results in Increased BMD and BV/TV when Compared to Controls.

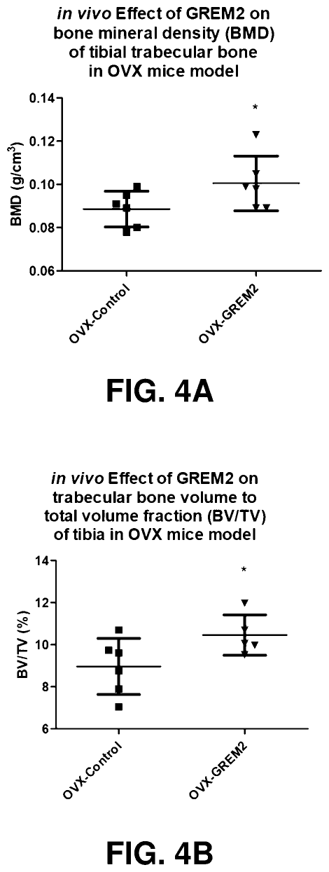

After treating the OVX mice with recombinant GREM2 for 4 weeks, it was found that BMD and BV/TV at the proximal tibia was significantly higher in the treatment group when compared to the controls. The mean difference was −0.012 g/cm³ (p=0.042) for BMD and −1.495 (p=0.033) for BV/TV (FIGS. 4A and 4B).

### CONCLUSIONS

In this study, the potential of GREM2 for the prevention and treatment of osteoporosis was evaluated. It was found that gene expression of GREM2 was causally associated with BMD in humans, and the circulating GREM2 was also cross-sectionally and positively associated with BMD. It was also found that circulating GREM2 was inversely associated with both bone resorption and bone formation markers, indicating that the increased BMD may be, in part, through inhibiting bone turnover. This is supported by the in vitro experiment showing that GREM2 treatment reduced osteoclastogenesis, and bone resorption in osteoclasts in a dose-dependent manner. Since age-related and postmenopausal osteoporosis is characterized by increased bone turnover, inhibition of bone turnover could improve age-related and postmenopausal bone loss.

The in vivo study confirmed that recombinant GREM2 treatment leads to increased BMD in healthy mice and OVX mice, strengthening GREM2 as a potential treatment for both age-related and post-menopausal osteoporosis. Taken together, these results show that GREM2 is a novel protein that can be used for the prevention and treatment of osteoporosis and bone loss. In addition, serum GREM2 can be a blood-based biomarker for osteoporosis and bone turnover.

Those skilled in the art will recognize, or be able to ascertain, using no more than routine experimentation, many equivalents to the specific embodiments of the method and compositions described herein. Such equivalents are intended to be encompassed by the following claims.

1. A method of treating a subject having a disease, disorder, or condition associated with bone loss, the method comprising administering to the subject a composition comprising:
(a) an effective amount of a soluble protein comprising all or a functional portion of amino acids 22 to 168 of a Gremlin-2 (GREM2) protein, or a functional variant thereof, wherein the function of the functional portion and functional variant comprises all or at least 20% of the native GREM2 protein antagonism to Bone Morphogenic Protein-2 (BMP2); or
(b) a nucleic acid encoding the soluble protein of (a), wherein the nucleic acid includes sequences mediating expression of the soluble protein in cells of the subject after the administration.

2. The method of claim 1, wherein the soluble protein comprises the amino acid sequence SEQ ID NO:2, or a functional variant thereof having an amino acid sequence at least 70%, 75%, 80%, 85%, 90%, 95%, or 99% identical to any one of SEQ ID NO:2.

3. The method of claim 1, wherein the amino acid sequence of the soluble protein comprises at least 70% and less than 100% sequence identity to any one of SEQ ID NO:2.

4. The method of claim 1, wherein the functional variant binds to BMP2 with at least 60% of the affinity of GREM2 protein.

5. The method of claim 1, wherein the functional variant includes the sequence CX₆QX₆CX₆NX₂CXGXCXSX₃PX₍₈₋₁₃₎CX₂CXPX₈TLXCX₍₁₅₋₁₈₎CXC (SEQ ID NO: 4).

6. The method of claim 1, wherein the nucleic acid is RNA or DNA.

7. The method of claim 6, wherein the nucleic acid is modified or unmodified oligonucleotide; selected from the group comprising antisense oligonucleotides, short interfering RNA, microRNA, peptide nucleic acids, locked nucleic acids, aptamers, and spiegelmers.

8. The method of claim 1, wherein the nucleic acid is mRNA.

9. The method of claim 1, wherein the nucleic acid comprises one or more expression control sequence(s).

10. The method of claim 1, wherein the nucleic acid is, or is encoded by, a vector or a transposon.

11. The method of claim 10, wherein the vector is a viral vector.

12. The method of claim 11, wherein the viral vector is selected from the group consisting of a lentiviral vector, an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector comprising a combination of any two or more of an Adeno-associated virus (AAV) vector, Herpes Simplex virus (HSV) vector, vesicular stomatitis (VSV) vector, or a human Bocavirus vector (hBoV).

13. The method of claim 10, wherein the vector is a nucleic acid expression vector selected from the group consisting of a plasmid, a cosmid, and a replicon.

14. The method of claim 1, wherein the composition is administered at an interval selected from the group consisting of once a day, twice a day, once a week, twice a week, three times a week, four times a week, once every two weeks, once every three weeks, approximately once a month, once every two months, once every three months, once every four months, once every six months, once every eight months, once every nine months, and once a year.

15. The method of claim 1, wherein the composition is administered once or more times a week for up to a period of 1, 2, 3, 4, 5, 6, 7, 8, 9, 10, 11, 12 months, or more.

16. The method of claim 1, wherein the subject is human.

17. The method of claim 1, wherein the composition is administered to a human subject at a dose of between 0.01 mg/kg body weight of the subject and 100 mg/kg body weight of the subject, inclusive.

18. The method of claim 1, wherein the composition is administered in an amount effective to reduce the expression of bone-morphogenetic protein (BMP) in the subject.

19. The method of claim 1, wherein the composition is administered in an amount effective to reduce one or more bone turnover markers in a subject.

20. The method of claim 1, wherein the disease or disorder to be treated is selected from the group comprising pre-osteoporosis, osteoporosis, osteopenia, bone fractures, and/or bone cancer.

21. The method of claim 1, wherein the composition is administered orally, by injection, or by infusion.

22. The method of claim 21, comprising subcutaneously, intravenously, intramuscularly, intraperitoneally, or intradermally administering the composition to the subject.

23. The method of claim 1, wherein the composition further comprises a pharmaceutically acceptable buffer, carrier, diluent, or excipient.


SEQUENCE LISTING (4 sequences)

SEQ ID NO: 1 - protein, 168 aa, Homo sapiens
MFWKLSLSLFLVAVLVKVAEARKNRPAGAIPSPYKDGSSNNSERWQHQIKEVLASSQEALVVTERKYLKSDWCKTQPLRQTVSEEGCRSRTILNRFCYGQCNSFYIPRHVKKEEESFQSCAFCKPQRVTSVLVELECPGLDPPFRLKKIQKVKQCRCMSVNLSDSDKQ

SEQ ID NO: 2 - protein, 147 aa, synthetic construct
  source 1..147: note = recombinant GREM2 protein
RKNRPAGAIPSPYKDGSSNNSERWQHQIKEVLASSQEALVVTERKYLKSDWCKTQPLRQTVSEEGCRSRTILNRFCYGQCNSFYIPRHVKKEEESFQSCAFCKPQRVTSVLVELECPGLDPPFRLKKIQKVKQCRCMSVNLSDSDKQ

SEQ ID NO: 3 - DNA, 4176 bp, Homo sapiens
  source 1..4176: mol_type = genomic DNA
agagctaagtctccgggctcctttcacatctgcaggagcagccagcctgggaccgcgcggcgcggggcgcgctctcgcctctcctgcaccctcagccggcgcgcttctcttatgggcgtctgctgcagtctggctgcggtcgaactgaaagcggcggcgggagaccaaacttagaccccgctgtggactagagaactcagagaaggcagagggagagggagagagagagagagaagggacccgaggaggaggcttccatcacgtcattgcaggatgttctggaagctttccctgtccttgttcctggtggcggtgctggtgaaggtggcggaagcccggaagaaccggccggcgggcgccatcccctcgccttacaaggacggcagcagcaacaactcggagagatggcagcaccagatcaaggaggtgctggcctccagccaggaggccctggtggtcaccgagcgcaagtacctcaagagtgactggtgcaagacgcagccgctgcggcagacggtgagcgaggagggctgccggagccgcaccatcctcaaccgcttctgctacggccagtgcaactccttctacatcccgcggcacgtgaagaaggaggaggagtccttccagtcctgcgccttctgcaagccccagcgcgtcacctccgtcctcgtggagctcgagtgccccggcctggacccacccttccgactcaagaaaatccagaaggtgaagcagtgccggtgcatgtccgtgaacctgagcgactcggacaagcagtgagcgccgggccggacgcagctcagccccgcgcgcgcccggccgctgggtggcgccgccgccgcctctgtccctgccctccgagcccactgtcacgctgcctggtgttccccctcagcagcaagcacttctcttagggctgacggtgtccttgtcacagacgtggatcgcaagtggagcttttgctgatgtgttcctgaccgaccccgggtcccacctgtgccccctaaaccaggcggtggatcaggcccgaaggaaaatgctggaaaaccaccaccgccacccagcagtcagagaggacctttcgaggtgatgcactgtaaatccggcattgatgtttccaccatgtggaaaagcaaggatttccctggccccgccctgcactccagctcgcccctttcccgcgcgaagtgaaggatgcgtcacttttctgagcggcccctcagacctctcctgctggtccctggacctctggctgggagcgctcattcttgtgactggcaggctgccctcggtcgctgctgtgatgaggatatgtgcaacctccataaatatgtcggtggacggggcgtctcctaataaacctgataccaagaagacaacttgagccacttggatcccgaagtgggcttcccgggaaacctcgagcagagcaggctacacaatcccccatcctcaagtccccaccgcctcttcttcctcactgtccccgccccctatttgtggactaaagatgaactctggtgtgcatgctattattgctgcaattagaatattatcacacacaaaggtctctatattaacgctggttttataagtgaccgtatattgtaaagagctgttaaaaagtattatagacctattcatgtattatttcacatgttttgactctggcttgcagcaccattcggagtaaggatgacagcaggcccagaaggtgtcttactcagaaaaaaaaatgcctggtcagcaattcactgtcatgcacctatttttagattgggcagggagatgggaggagtcgtgatttttactttgaatactatttcactgacgttactaagcattgcagcacaatgtagaaattggcttgggatggataggtatagggaagggcattcgttgggaaattgatgaccggaaactagaaagcccctataatgtggaatatgcatatgtggggtgaaaccgtatactactttcactgccatgacactaggcaaaaaatatttcctgttaaaagaaagagaaaaagagagggatattgaaaaatataaggagttgagtcgctttgtggtatagaagggagtaaggaataaaagatatagaatagtaatctagttgaactgttctgcacaaaacatgcttccttttcaaagaatagacttcaaaagggacgaacaaacattaggtcaccttttgtgtttataatgaattgttgacgtttccatgctgtggtctcttgatatacggaagtagacgtaacttattttccagcgaaattatatacttcttctgggctcctttgctcagttgccccaggaaggcttccctgtgagtaatgcttatggaatctgacaaatgtgaatgagctgccattggtgggaatcagtaaactttgaaaacctttcactgatgattccctggtaagtgctcctgtgaggggccacttccctagtatgtgagagagaccctctgcaagccgctgtggggacttagcttcctgatactaggtagtgatggtggatctagagacattcacttgaagagcaaggattggggtggggtgggttggtgaatctatctcaggccactgcactgcaaggtgcatttctgtcatttctgcaacaccatgagacctgagcaagaatgtgctgtggcagagggggtcttggtttcataaatgggtatatacagcctgaactcaggaggcagaaatcaaaagaaatccttgagggctggaacctgaggatagagcgaaaatggagagaagggtgttcaaggtcaggagtgatttttgcatttggtggaaaacctcaagtcagactcagtggcagaacatgcagcagaaaagctgtgaaggaaggaaatttacgtgattgtaatggaggctaggggtggattgggaggtgagtggaggagagtctctgaccagctgcagagccaacccctatcattttattaacatggaacccacacagataggggagggattaagctaagaacttacagaatgcaaacacgagcacactctcttcgaacccaattgtgggtgtagcaatgaaagcaatatgatatgctgcagtgtgaagctccttctggggttatcgtatgtacaaagtttaccttataatggctcaaattgtatttaatttttttgttttgtttataaatgaagaaaagctataagtataatgtaattattttataggtatactattaaattatagaacaaataaagataatctaggattatatgtgatcccggtgtgacgtgccataaaagtggaatttttgacttgttgcagtgagctgtgaagcagcactatgcgttcagagagtttttgttcaaccatggtgagttcaacctctagcttatgtcacatgatctcctttggggtctggaatcttctaagagaaatagaatcaggcatccaaggagcagaagtgatggaggtggccctcaatcctggtctttggaagcttccaagcctcttccacatcccctgccagctgaagacagagtattctcactgtgctggctgaaggagaaacactaaacaaggaaaccttgagaaactgcagcaatggaagtccactgttagtctctccgttagcaccagggacacacttgttctgagttttgttcattttcacatttactcctcatgagttccaacatttcttaatttaaatatttcttagtttatagcatctccatttttctattaattgacttaatctgccatttaagaaaactccgtaatcaataaaaaataaaatccacaatcaaactgcacatggttgatccactgtgtaggcttcaaaggaattaaactcaacttcctaggaaataattcatcttagtttattaaatgggacagaatatttcactgctaacgtattctaactcacttgtttttgaaattggccagatgatttttaagttataaagtcaatgaaaatgacttttgctattgtgtgaacaacttagaatgtatatattcccagaaggcttatttaatgattgacctatcaccactgattattaatgtctgcatgaatgataacaataagaccaagtcatcaataatgcaatgaaaattttttaagtgaaagcaagtttattaagaaagtaaaggaataaagaatgcctacttcataggcagagcagcctaa

SEQ ID NO: 4 - protein, 87 aa, synthetic construct
  source 1..87: note = consensus sequence
  VARIANT 2..86: note = X is any amino acids
  VARIANT 44..48: note = at most five X can be deleted
  VARIANT 81..83: note = at most three X can be deleted
CXXXXXXQXXXXXXCXXXXXXNXXCXGXCXSXXXPXXXXXXXXXXXXXCXXCXPXXXXXXXXTLXCXXXXXXXXXXXXXXXXXXCXC